(19) United States
(12) Patent Application Publication
(10) Pub. No.: US 2023/0274798 A1
(43) Pub. Date: Aug. 31, 2023

(54) METHODS OF ANALYSING OR GENERATING SEQUENCES OF ENCODING ELEMENTS

(71) Applicant: GGF Global Genomics Pty Limited, Hornsby (AU)

(72) Inventor: Christopher Charles Hagan, Waverton (AU)

(21) Appl. No.: 18/072,417

(22) Filed: Nov. 30, 2022

Related U.S. Application Data
(63) Continuation of application No. PCT/AU2021/050540, filed on Jun. 1, 2021.

(30) Foreign Application Priority Data
Jun. 1, 2020 (AU) 2020901794

(51) Int. Cl. G16B 45/00; G16B 30/00; G16B 35/00

(52) U.S. Cl. CPC G16B 45/00; G16B 30/00; G16B 35/00

(57) ABSTRACT

The invention relates to a sequence analysis method or sequence generation method for analysing or generating sequences of encoding elements of a particular DNA, RNA, or macromolecule sequence. The invention also relates to a manual, automated or computer implemented method of recognizing, analysing, probing, testing, processing, sequencing and sensing DNA or other macromolecular sequences.

### CROSS-REFERENCES TO RELATED APPLICATIONS

This application is a continuation application of International Patent Application No. PCT/AU2021/050540 entitled “METHODS OF ANALYSING OR GENERATING SEQUENCES OF ENCODING ELEMENTS,” filed on Jun. 1, 2021, which claims priority to Australian Patent Application No. 2020901794, filed on Jun. 1, 2020, each of which are herein incorporated by reference in their entirety for all purposes.

### SEQUENCE LISTING

The instant application contains a Sequence Listing which has been submitted electronically in XML format and is hereby incorporated by reference in its entirety. Said XML copy, created on May 23, 2023, is named 109587-005700US-1355553_SL.xml and is 20,308 bytes in size.

### FIELD

The invention relates to a sequence analysis or sequence generation method for analysing or generating sequences of encoding elements of a particular DNA, RNA, or macromolecule sequence. The invention also relates to a method of recognizing, analysing, probing, testing, processing, sequencing and sensing DNA or other macromolecular sequences to produce motifs, images signatures or other representations (“GGF Motifs”) and their application to recognition, analysis, probing, testing, sensing, modelling, formatting, classifying, cataloguing, statistical analysis or manipulation, or physical testing by other means of analysing such sequences for the purpose of detecting images, configurations, patterns, graphic signatures or representations of code execution or encoding or other code mapping or reverse mapping which may be inherent in the order of such Code Sequences. The invention also relates to a medium accessible by a computer storing program instructions for execution on a computer system which when executed by the computer instructs the computer to execute the method.

### BACKGROUND OF THE INVENTION

The completion of the human genome project has opened up many new areas of research, development and clinical applications involving biological, biomedical, biochemistry, bioengineering, bioinformatics, biomathematics and computer science. In particular, Artificial Intelligence (AI) systems based on genome logic and Next Generation Sequencing (NGS) is allowing greater analytical penetration in the human genome to better understand factors behind disease and allow precision medicine such as genetic testing to test an individual's propensity to disease and genetic engineering to create measures to combat such propensities or treat such disease.

Research techniques and resources which have benefited include genetics, cancer and disease research and biology such as evolution and origin of life research. Research to develop clinical use relies heavily on genetic analysis and genetic libraries or databases of all kinds.

Clinical applications include genetic screening for known genetic sequences prone to cancer or other disease as an aid to treatment or pre treatment. Genetic analysis has improved with the compilation of various genetic libraries which can be used for both research and clinical use.

However, bacterial and viral research, development and clinical applications remain important. Research on infectious diseases and methods to combat those often hinge on genetic analysis of the bacteria and virus and the molecular design of drugs or other treatments to stop bacterial or viral spread. The recent Coronavirus outbreak underscores the importance of this work.

At the same time viruses and bacteria can be put to therapeutic use and again their genetic sequences play a central part in developing these usages.

However, the great challenge is the analysis of the genetic code itself and its scheme of expression for gene transcription leading to protein synthesis. Thanks to work on such genomes as Drosophila melanogaster and discovery of such master gene networks as Hox genes (highly conserved over evolutionary time) conceptual theories of how the genetic code works are emerging but there is a long way to go.

The discovery of the basic triplet code—codons—which transcribes triplet codons to the canonical 20 amino acids was a major step after Watson, Crick and others discovered the structure of DNA.

The fact that DNA is not only a macromolecule but is variable such as to contain a code is striking. However, beyond the discovery of the triplet code/codons, the high-water mark of knowledge about how the DNA code actually works has been the mapping of gene sequences—exons or genes in pieces matching the fate of gene transcription to their various destinations or in the case of defective genes the abnormality resulting from the transcription of those defective genes.

The discovery of repeater sequences, exons, introns, palindromic DNA and the many other ‘structural’ or statistically based discoveries do not provide a ‘Rosetta Stone’ as to how the code works. Its language is still foreign to us despite seeing it in action and its various outcomes.

Bioinformatics uses image and signal processing to analyse large amounts of DNA or protein sequences from genomes or proteomes using pattern recognition, data mining, machine learning and statistical visualization methods. Such data is then analysed and interpreted to provide databases and libraries with predictive power. Bioinformatics allows sequence detection, gene matching, comparison, sequence analysis, classification and many other interpretation techniques for genomic data. This occurs through the development of gene or protein ontologies used for genetic and clinical research. Many bioinformatics scientists are engaged in mapping and analysing various genome and proteome sequences using these analysis techniques to better understand biology and create ontologies which will aid future biological and medical research.

Pioneers in the field included Fred Sanger who sequenced insulin in the early 1950s and Elvin Kabat and Tai Te Wu who pioneered biological sequence analysis. For example, the Wu Kabat Variability plots produced statistical graphs showing the relationship between gene expression and proteins and allowed DNA sequences to be analysed—for example, whether the sequence was random or non random. Margaret Oakley Dayhoff constructed one of the first protein sequence databases, sequence alignment methods and molecular evolution methods.

It is to be understood that if any prior art publication is referred to herein, such reference does not constitute an admission that the publication forms a part of the common general knowledge in the art in Australia or any other country.

### BRIEF SUMMARY OF THE INVENTION

According to a first aspect of the invention, there is provided a sequence analysis method for analysing sequences of encoding elements of a particular DNA, RNA, or macromolecule sequence, comprising the steps of providing a sequence data file defining an ordered collection of encoding elements, each being one of a plurality of encoding element types, formatting the sequence data file to generate a formatted data file, wherein the formatted data file corresponds to a representation of the sequence data file according to one or more user-defined and/or pre-defined formatting parameters, the formatted data file defining an ordered set of encoding elements, determining an angle set defining, for each encoding element type, a corresponding angle in an n-dimensional space (n>1), wherein each angle may be defined in polar co-ordinates, the determination based on one or more user-defined and/or pre-defined angle generation parameters, recursively and in order, applying the angle set to the formatted data file, thereby generating a mapped data file, said mapped data file defining a set of points in the n-dimensional space and linkages between adjacent pairs of points, displaying and/or storing the mapped data file, wherein the mapped data file is configured to enable generation for display of a visual representation of the relative locations of the points in the n-dimensional space and the associated linkages.

According to a further aspect, there is provided a sequence generation method for generating sequences of DNA, RNA, or macromolecule encoding elements, each being one of a plurality of encoding element types, comprising the steps of providing a spatial data file defining a measured or desired spatial representation of a biological sample, determining a profile of the spatial representation in an n-dimensional space (n>1) according to one or more user-defined and/or pre-defined profile parameters, determining an angle set defining, for each encoding element type, a corresponding angle in an n-dimensional space (n>1), wherein each angle may be defined in polar co-ordinates, the determination based on one or more user-defined and/or pre-defined angle generation parameters, utilising the angle set to identify a predictive data file defining sequence of encoding elements, wherein an initial position in the profile is selected and an outline of said profile is generated by recursively identifying particular encoding elements based on a best-fit identification of a next angle selected from the angle set such as to optimise a similarity between the profile and the outline; and storing the predictive data file.

A further aspect provides a manual, automated or computer implemented method or methods including all possible configuration of methods as described in this application (“Methods”) of recognizing, analysing, probing, testing, processing, sequencing and sensing DNA or other macromolecular sequences by a party or parties, consisting of the steps of the entering into a source dataset, database or library the code sequences of the DNA of a genome or other code sequences of other macromolecules (“Code Sequences”), formatting the elements of such Code Sequences and then applying an algorithm to such elements by selected extraction procedures to produce co ordinates in two or more dimensions or phase spaces as required for representation in graphic, geometric, algebraic, topological or other mathematical form whether paper based, digital or using other means of processing and storage to produce motifs, image signatures or other representations (“GGF Motifs”) to conduct recognition, analysis, probing, testing, sensing, modelling, formatting, classification, cataloguing, statistical analysis or manipulation, or physical testing by other means of analysing such sequences for the purpose of detecting images, configurations, patterns, graphic signatures or representations of code execution or encoding or other code mapping or reverse mapping which may be inherent in the order of such Code Sequences in order to interpret and provide systematic representations of the Code Sequences analysed with relevance and for use in bioinformatics, biological, biomedical, medical, veterinary, biochemical, biotechnological, pharmaceutical fields, vaccine development, genetic testing and allied sciences and applied sciences (or related fields) whether for research, development, clinical, commercial, industrial or other uses.

A further aspect provides a medium accessible by a computer storing program instructions for execution on a computer system which when executed by a computer instructs the computer to execute a series of commands which constitute the steps for implementing the method of the first aspect including such medium that in the form of computer code for use on its own or with other programs or a medium in the form of operative code within DNA or other macromolecular sequencing and analysis computer programs in general use by biologists, clinical, medical, scientific, commercial or other staff in the said fields set out in the first aspect which programs are used with or without access to datasets, databases or libraries.

The method described below, the Generative Genome Function (GGF), is a bioinformatics tool that allows DNA sequence analysis and visual representations using dynamic data processing, formatting and recursive algorithms utilising sequence formatting, geometric algorithms, parameter setting and graphical representations with computational biological tools.

It is a new method designed to extract and recognize patterns related to the geometries of DNA that may reflect transcription outcomes to portray or map DNA sequences visually which will allow new ontologies of DNA or protein structure to be created. The GGF method is intended to allow new techniques of image and signal processing to be utilized to enhance existing DNA or protein libraries or databases which have higher recognition signatures amenable to better classification methods and query systems.

GGF images and motifs can provide superior signatures and motifs to existing graphical representations of DNA or other coding macromolecules which are designed to greatly improve interpretation, analysis and understanding of genomic data.

For example, GGF produces meaningful images in non coding intergenic zones of DNA providing new images that could provide a basis for discovering new gene expression mechanisms, evolutionary insights or epigenetic features otherwise unseen. The Wu Kabat variability plots can gauge non randomness whilst the GGF can not only indicate non randomness but produce meaningful motifs or signatures in clear images that can include generic motifs that may lead to matching other sequences which may be otherwise be difficult to match e.g. where substantial ‘noise’ obscures a generic or matched sequence. This is because GGF is not matching similar DNA sequences but producing motifs or signatures that can be compared. These sequences may be quite different but the image of the motifs are similar giving perhaps the only way to link the two loci of DNA under study. Thus, the GGF can provide solutions to the increasing problems of massive amounts of raw code derived from sequences.

To take one example of a graphical method which specifically analyses a type of DNA sequence representing clusters of non coding RNA sequences, the method is described in an article: Heyne S, Costa F, Rose D, Backofen R. GraphClust: alignment-free structural clustering of local RNA secondary structures. Bioinformatics. 2012 Jun. 15; 28(12): i224-32. doi: 10.1093/bioinformatics/bts224.1. In that article the authors refer to applying their Cluster graph method to >220,000 sequence fragments to obtain a “small number of probable, but sufficiently different, structures for each RNA sequence. We then encode each structure as a labelled graph preserving all information about the nucleotide type and the bond type . . . in this way a sequence's structure is represented as a graph with several disconnected components. We could now compute the similarity between the representative graphs using a graph kernel.” By use of various matching techniques such as ‘nearest neighbour’, covariance and refinement analysis and so on matches between the sample sequence and the target sequence can be made.

The GGF method has a new inventive step beyond methods such as GraphClust because whilst the GraphClust is a statistical visualization method designed to produce graphical non recursive representations merely aiding statistical correlation between the sample sequence and the target sequence, the GGF method is more than a statistical visualization method in that it contains recursive, geometrical and formatting features designed to model actual possible biological processes such as the possible manner in which the recursive generation of polypeptides which fold occur or the possible manner in which micro or macro molecules tile into a macromolecule or tissues of the body and so on. Generating processes in the genome and proteome are known to be recursive and so recursive representations can better model biological processes.

The examples below provide evidence of the success of the method in modelling actual biological patterns that may be intermediate or final phases in generation of macromolecules or morphogenesis.

Thus, the central aim of bioinformatics to better understand biology for both biological and medical research is greatly empowered by a method which is designed to better model the biological processes that are outcomes of code execution in genomes or proteomes. At the same time the serious problem of the noisy raw data clouding analysis of code sequences may be lessened by the GGF technique because of its demonstrated ability to produce smooth, recognizable, unique motifs and signatures across any DNA sequence including intergenic sequences.

The GGF method the subject of this application processes a biopolymer—optionally DNA—including a sequence of bases in DNA or RNA or other macromolecules including protein sequences such as amino acids in polypeptide chains or proteins. (“GGF” refers to “Generative Genome Function”). The present working version of the GGF algorithm used in the GGF method has been written in both Basic and Mathematica (preferably Mathematica) but could be written in other computer languages if required. The GGF method includes the GGF algorithm which converts a formatted linear sequence/chain of bases/molecules into a series of cartesian co ordinates preferably in polar format which are then mapped to become an image called a “GGF Motif” which is postulated to represent a visual representation of molecular structure in a topological and/or morphological sense but may represent other mathematical features or relations yet to be discovered. For example, the GGF Geneseeker method has been applied to the G4 Bacteriophage genome of 5577 bases to produce a series of cartesian co ordinates in polar form which are mapped to a cartesian graph to produce a GGF motif which is considered to resemble the body plan of the Bacteriophage (See FIG. 1). A further example, involves applying the GGF method to more complex genome sequences en globo, specific sequences or gene fragments (exons) or both exons and introns to gauge the general nature of the sequences to classify them.

The GGF method has a number of uses:
(1) A recognition tool, analytical probe, test, search or sensor tool to analyse or search DNA, RNA, proteins or polypeptides or other macromolecular sequences in databases or otherwise to detect images, configurations, patterns, graphic signatures or representations of code execution or encoding or other code mapping or reverse mapping which may be inherent in the order of such macromolecules in order to interpret and provide systematic representations of the particular macromolecular code analysed with relevance and use for use in bioinformatics, biological, biomedical, medical, veterinary, biochemical, biotechnological, pharmaceutical fields, vaccine development, genetic testing and allied sciences and applied sciences (or related fields) whether for research, development, clinical, commercial, industrial or other uses and which can take the form of computer code for use on its own or with other programs or take the form of operative code within a biopolymer including DNA or other macromolecular sequencing and analysis computer programs in general use by biologists, clinical, medical, scientific, commercial or other staff in the said fields which programs are used with or without access to datasets, databases or libraries;
(2) As datasets, databases or libraries;

Different biopolymers DNA motifs, DNA sequences or DNA alphabets for different biopolymer including DNA sequences can be compiled into a library or database of GGF motifs which could be compiled into a legend or table with GGF Motif elements matching DNA sequences, motifs, alphabets, genes, morphologies, exons, introns, mutations, neoplasms, metaplasias, irregular proteins, isoform proteins, dysfunctional proteins, or other genetic or biological features or abnormalities or defects.

This legend or table could form a type of ‘Rosetta stone’ allowing valuable interpretation of information with respect to DNA, RNA, polypeptide or other sequences, gene transcription fate maps, gene regulation maps, gene circuits, protein circuits, metabolic maps, or other biological circuits, protein synthesis and folding analysis, cell signalling, cell differentiation, morphologies or a general scheme for different gene expression propensities for different types of gene expression for research, development, clinical or commercial use.

The uses of such libraries or databases could include: Compiling libraries or databases including new genomics, bioinformatics or other databases to classify target genetic or other sequences to search for, detect or produce information, models and other research/development tools, genetic defect risk profile database or a new genetic parts database or RNA interaction library or other libraries or databases for use in bioinformatics, biological, biomedical, medical, veterinary, biochemical, biotechnological, pharmaceutical fields, vaccine development, genetic testing and allied sciences and applied sciences (or related fields).

(3) As a design tool by the use of GGF motif libraries, databases or elements or statistical methods or libraries, databases or elements which utilize GGF Motifs to design, generate, calibrate, fine tune or otherwise supply DNA/RNA, amino acid or other sequences to fulfil metabolic, morphological, cell differentiation or cell signalling functions, genetic, protein or other circuits (designed or otherwise), produce therapeutic products, treatments, or tools for use in bioinformatics, biological, biomedical, medical, veterinary, biochemical, biotechnological, pharmaceutical fields, models, medicine or chemicals, facilitate genetic engineering, genome editing, tests, sensors or other biological products or procedures by various methods.

Thus, the GGF method could form the basis for new discoveries by GGF research programs or mere use in clinical or commercial settings where results are analysed scientifically. The GGF method also provides a way of ‘smart testing’ DNA or RNA sequences which can be compared to statistical analysis whether for the purpose of gauging type of sequence—random or non random and many other relevant features. Such test can be used on its own or with other algorithms, statistical analysis, data mining, AI programs or as a primary tool for compiling libraries, models, circuits, tuning calibrations or other databases. It is considered that the GGF method can become a new important tool for research, development and clinical use because it provides libraries or databases which when combined with other DNA-RNA-protein libraries can be cross linked to yield new relationships, insights and even direct discoveries whether by direct analysis, GGF comparison testing or statistical correlations or other new GGF motif based statistical techniques. It can improve data mining effectiveness and reduce running time of DNA analysis—especially high throughput analysis by reducing target sequences to sub sequences more quickly depending on the GGF library or model that has been created or may by direct analysis provide indicators for fine tuning design of genome data mining tasks or projects. The GGF method can form the basis for the formation of other methods to be used to generate synthetic DNA, RNA or protein sequences or to aid existing algorithms designed for those tasks.

### BRIEF DESCRIPTION OF THE DRAWINGS

FIGS. 1A and 1B. GGF PRINTS OF BACTERIOPHAGE G4

Two GGF print images resulting from application of the GGF method to the 5577 bases of the G4 Bacteriophage. FIG. 1A uses the standard GGF algorithm and formatting where the GGF prints symmetry by reversing the order of DNA letters. FIG. 1B is an alternative GGF print involving a different base order to explore whether the phage DNA is capable of expressing different types of phage morphologies.

FIG. 2. REAL DROSOPHILA GENOME CODE GGF PRINT VS RANDOM GGF PRINT

GGF prints conducted to test whether the GGF images were merely mathematical curiosities (for example, like fractals) or whether a real meaningful function like effect was the product of the DNA code itself. Clearly the 72,000 random letter print is a truly random curve and not smooth, meaningful nor function like. Thus, it suggests the intergenic sequences i.e. exon—intron stretches are non random as a number of authors suggest and include intergenic regions which are not ‘junk’. Left figure caption: Drosophila Melanogaster >72,000 bp [File ref. FBGN0000179—Bi gene representing ventation of wings. Right figure caption: 72000 random letters consisting of A, G, C and T were processed to produce a GGF print with the GGF algorithm.

FIG. 3. Z⁵−1=0 DNA SYMMETRY

Selection of {r1, r2, r3, r4}˜{2,4,6,8} in the GGF Algorithm produces what is referred to as a pentagonal GGF because the co ordinates produced by the mapped angles represent a pentagon on an Argand diagram where the 5 points of the pentagon are the roots of unity of the equation, Z⁵−1=0. Note that A & T can be exchanged relative to C and G depending on configuration of GGF algorithm chosen.

FIG. 4. VARIOUS GGF MOTIFS & DROSOPHILA COMPARISON

The GGF method was applied to the Human Mitochondrion DNA (>16,000 bp) and compared to a random A, G, C & T GGF print (>16,000 bp) (left). The middle diagram allows the GGF print for Drosophila melanogaster fruit fly (>72,000 DNAbp) to be compared to the venation (framework) of wing.

FIG. 5. GENERIC GGF MOTIFS—ALPHA HELICES & BETA SHEETS

The DNA sequences of 2 proteins were both subjected to a GGF print. On the left a GGF print of Glycophorin (alpha helix shaped proteins that span a cell membrane) and on the right a GGF print of Porins (beta sheet shaped proteins that form pores across cell membranes). Thus the GGF produced a motif of helices for Glycophorin and a ‘sheet of dots’ motif for Porins.

What is striking is that on reversing direction of GGF code the Porins gene printed a helical cord with some similarity to the Glycophorin gene's normal helical print whilst Glycophorin's reverse print was a ‘sheet of dots’ patterned not unlike the dots in the Porin's beta sheets motif. Equally striking was a GGF frame test performed on both Bacteriophage G4 virus and Covid-19 virus both of which displayed similar generic GGF motifs namely ‘sheet of dots’ motifs on one base GGF frame shifts possibly indicating similar generic transcription schemes in each viral genome encoding key generic elements of viral proteins, similar to Porins Beta Sheets—See Panel 3, FIG. 5.

FIG. 6 shows the output of Attachment A of the Examples.

FIG. 7 shows the output of Attachment B of the Examples.

FIG. 8 shows an output of the methods according to the invention, corresponding with a ‘seahorse’ strand that when combined with similar strands might produce a ‘pocket’ (target receptor) on the phage virus protein to enable ligand design. The figure illustrates a GGF motif printed from Gene 9 Phage p22 tailspike compared to its protein.

On the left, a GGF motif (>2000 bp) of Gene 9 encoding the Salmonella Phage p22 Tailspike protein—which has parallel beta helices. It is postulated that the GGF motif above represents the essential topology of the nascent polypeptide chain (right). On the right, the FIG. shows unfolding of nascent polypeptide chain of the Phage p22 Tailspike protein From Protein Folding Failure Sets High-Temperature Limit on Growth of Phage P22 in Salmonella enterica Serova Typhimurium Welkin H. Pope, 1,2, Cameron Haase-Pettingell,1 and Jonathan King1 APPLIED AND ENVIRONMENTAL MICROBIOLOGY, August 2004, p. 4840-4847 by permission of the American Society for Microbiology.

FIG. 9 shows an example GGF Codeweaver output of Stage 1. The green dotted profile generated by GGF Codeweaver to fit the orange dotted profile of the receptor. The inset graphical illustration includes the labels ‘The central 10 orbit letters are chosen plus similar flanking sequences’, ‘Ligand profile generated’, and ‘Receptor profile’. On the left hand side are a list of angles captured from receptor profile used to generate DNA letters using GGF. On the bottom is the DNA sequence captured from receptor profile and used to generate a GGF ligand profile. Part of the DNA sequence is a section labelled ‘10 orbits+like orbits’. Below that section are coordinate X values and Y values for ligand profile.

FIG. 10 shows schematics of the GGF Geneseeker and GGF Codeweaver according to embodiments of the invention. The top schematic illustrates the GGF Geneseeker—GGF map formatting. Think of the culling of each 14 letters like loops before they are repeated 15 times to replenish the sequence. The GGF algorithm formats the raw DNA into a ‘sparse ten repeater’ format. Figure discloses SEQ ID NOS 1-6 and 5, respectively, in order of appearance.

The bottom schematic illustrates the GGF Codeweaver—GGF inverse map formatting. The GGF walk profile theoretically produces the extrapolated DNA sequence. When the GGF walk profile is constructed, we assume it is in this sparse ten repeater format which is sampled and then populated with missing ‘in between’ DNA letters that encode the expected peptide chain. Now our GGF walk profile is the output of a chain of vectors of T, A, G, T . . . being added so each 10 letters should theoretically appear 15 times in our GGF walk profile but ‘noise’ in that process prevents a fully deterministic sequence so a process of sampling is adopted. This sampling to extrapolate back to the sparse ten repeater sequences aims at generating the sparse orbit bases & so: 1. Sample 10 letters in 10 frame shifts, 2. The first samples are analysed by Stage 3 of the Codeweaver algorithm as if those 10 letters are the original sparse orbit bases, 3. The best segment of 10 to be a candidate ligand is selected which can be extended at either end of that 10 sequence if the flanking sequences repeat or are similar to that sequence, 4. Then the ‘in between’ missing peptides are populated (i.e those encoded by the full unknown DNA sequence being extrapolated).

### DETAILED DESCRIPTION OF THE INVENTION

### A. General Description of the Utility of the Method

A.1 GGF Geneseeker

The GGF method, in embodiments the GGF Geneseeker, allows biologists, medical scientists and in particular bioinformatics scientists a new tool to face the great challenges to process, analyse, classify and utilize the large volumes of genome, proteome and other data despite the serious impediment of separating meaningful data from noise.

In particular, existing techniques of base alignment matching, pattern or signal recognition and the many algorithms seek to recognize gene or other motifs to classify, analyse and store. The GGF uses a series of steps including DNA formatting, application of an algorithm, parameter setting and graph representations to allow more motifs to be recognized via graphical representation to produce a geometrical generated graphical representation. This new technique of genome or proteome based image or signal processing using dynamic recursive algorithms, formatting and parameter settings is designed to allow:
(i) higher success rates of motif or other target sequence recognitions;
(ii) more classes or generic motifs that can be utilized in genome or proteome recognition tasks by use of GGF libraries and ontologies that the user or the biological community can create as reference databases to aid the recognition of GGF motifs, images and signatures that represent known elements in such libraries or databases;
(iii) better classification and query systems for libraries and databases by reason of the increased repertoire of GGF motifs, images and signatures including generic GGF motifs, images and signatures;
(iv) potential for reverse GGF processing and modelling to seek new genomic or proteomic motifs, images or signatures for genetic or proteomic engineering or artificial intelligence bioinformatics systems.

A.2 GGF Codeweaver

The GGF method is intended to reproduce the shapes, patterns and morphology of proteins, cells, tissues etc. which involves mapping a DNA or macromolecular sequence to a graphical representation of those shapes patterns or morphology. For convenience, the use of the forward version of the GGF method ie mapping from DNA to image shall be now referred to as GGF Geneseeker. The inverse of the GGF method—inverse mapping from image to DNA sequence shall be referred to as GGF Codeweaver.

In the field of biosynthesis design of synthetic molecules designed for a specific industrial, medical or other purpose. Industrial enzymes and biopharmaceuticals are two major areas of endeavour driven by advances in genetic techniques such as drug design of biologics/biosimilars, genetic engineering, metabolic engineering and molecular breeding techniques such as directed evolution of aptamers. Genomics, proteomics and metabolomics provide knowledge databases for designing industrial enzymes and small molecules for medical use.

Microbiologists (‘biologists’) design biosynthetic products eg, an industrial enzyme, a new antibody or drug to fight cancer, disease or viruses using bioinformatics databases, computational design tools, wet lab methods etc. A core aim in designing the synthetic molecule is to conceptualize the shape, structure and charge that the molecule should take. In the pharmaceutical industry ‘drug like molecules’ ie that resemble drugs or contain features similar to existing drugs can guide the design of a pharmacophore ie a model drug. Thus, shape, structure and (indirectly) charge can be greatly aided if a visualization method can guide existing design methods which are already challenging. Where can GGF Codeweaver aid design in these biosynthetic industries? The shape, structure and charge of a drug molecule are decisive of the function the drug is to play in the biological sense. For instance, in drug design a small molecule called a ligand is designed which inhibits or activates a biomolecule (or turns genes on or off) giving therapeutic benefit to the patient.

Thus, it would be of great utility for the biologist to ‘draw’ the shape of macromolecule that he or she wishes to design. So, again taking drug design, where the small molecule (drug) is a ligand and the target biomolecule to be changed is a ‘receptor’ then the docking of ligand to receptor becomes a ‘key and lock’ problem where shape is paramount. Structure and charge are also paramount (a ligand is a molecule that binds to a biomolecule generally donating electrons to become bound to it for a biological purpose—the biologist will either want to inhibit or activate that purpose).

Thus, if the biologist creates a profile of a receptor surface GGF Codeweaver can take a shadow profile and apply the inverse algorithm to generate possible amino acid or DNA sequences that could make up that model ligand or generate it.

Thus, GGF Codeweaver could then help design biosynthetics for:
Oligopeptides to modify disease causing proteins
Ligands or pharmacaphores for drugs or vaccines
Finding DNA variants to inactivate deleterious genes or promote good ones
Gene therapy
Immunotherapy
Other biosynthetic products—medical, biological or industrial to create biosynthetics or biosimilars by designing ligands eg drugs for medicine or enzymes for industry in combination with such techniques as virtual screening, rational drug design or protein evolution.

### B. Potential Uses and Examples of the GGF Method

B.1 GGF Geneseeker

The present GGF method brings us a step closer to decoding the language of DNA—it provides part of the Rosetta stone. Why? In embodiments of the GGF method when applied to DNA sequences produces images—called GGF motifs—which produce different motifs for different sequences appearing with hieroglyphic likenesses awaiting a Rosetta stone. Already GGF is forming a library of these motifs and some occur more often such as spirals, helices or sheets or distinctive dot patterns which could qualify as ‘generic GGF motifs’.

There is evidence that these motifs are more than beautiful shapes or patterns. A GGF printout of the common G4 Bacteriophage genome (5577 base pairs) produces an image or GGF motif which resembles the geometric body plan of the bacteriophage—its morphology. This suggests in this instance the GGF method has decoded the raw DNAcode transcription, through protein synthesis directly into the patterning and layout of those proteins into the body plan which resembles a microbial mosquito—indeed the bacteriophage virus injects its genetic material into bacteria like a mosquito (except a mosquito does not inject, it sucks.) In a planned publication (book or paper) after this application, a hypothesis is proposed as to why the GGF motif from a bacteriophage (presumably ‘exons only’) produces apparent morphology yet GGF motifs from intergenic regions of more complex genomes still produce well formed shape images (e.g. applying the GGF to an intergenic genome segment of Drosophila melanogaster fruit fly still produces beautiful motifs, shapes and patterns interpreted as venation (framework) of the wing.) Although most complex DNA sequences are not so readily interpreted they have been postulated to mean that the GGF is decoding vast exon—intron regions of DNA representing a repertoire of working modules/nascent/candidate genes or even so called non coding housekeeping genes (which may actually be nascent codes or real codes). In contrast, the bacteriophage is typically ‘exon’ only and thus it seems only genes are decoded with recognizable visual output by the GGF and are thus expected to show direct outcomes of transcription. With the more complex genomes motifs are not irregular indicating persisting biological meaning and structure (not junk) and it has been postulated that the GGF is ‘decoding’ both genes and genes in development and that is why GGF motifs are still well structured shapes and patterns—like a cascade of potential morphologies. Theory aside, the GGF method is superior to existing DNA sequencing analysis because it produces a suite of visual generic motifs, patterns and shapes in many cases and in the balance of cases still produces non random visual shapes and patterns sufficient to identify that signature as a ‘GGF motif’ for that sequence. Thus, the GGF method has a great role to play in NGS, research and industry.

Thus, it is considered that the GGF method can become an important tool for research, development and clinical use because it can provide libraries or databases which when combined with other DNA, RNA—protein libraries can be cross linked to yield new relationships, insights and even direct discoveries whether by direct analysis, GGF comparison testing or statistical correlations which might include new techniques such as ‘regression to the GGF Motif’ as an improved replacement to ‘regression to the line’ analysis (or similar techniques).

The GGF Geneseeker method in embodiments of the invention processes a sequence of bases in DNA or RNA—optionally DNA—(and possibly amino acids in polypeptide chains such as in proteins) that was, in an earlier form, originally authored in BASIC by Dr BE Hagan (deceased 2007). This algorithm used in embodiments of the invention has been called the Generative Genome Function or Geneseeker—in short the GGF Geneseeker. The present working code version of the GGF Geneseeker embodiments and algorithm has been written in Mathematica (Author—CC Hagan, the present inventor applicant).

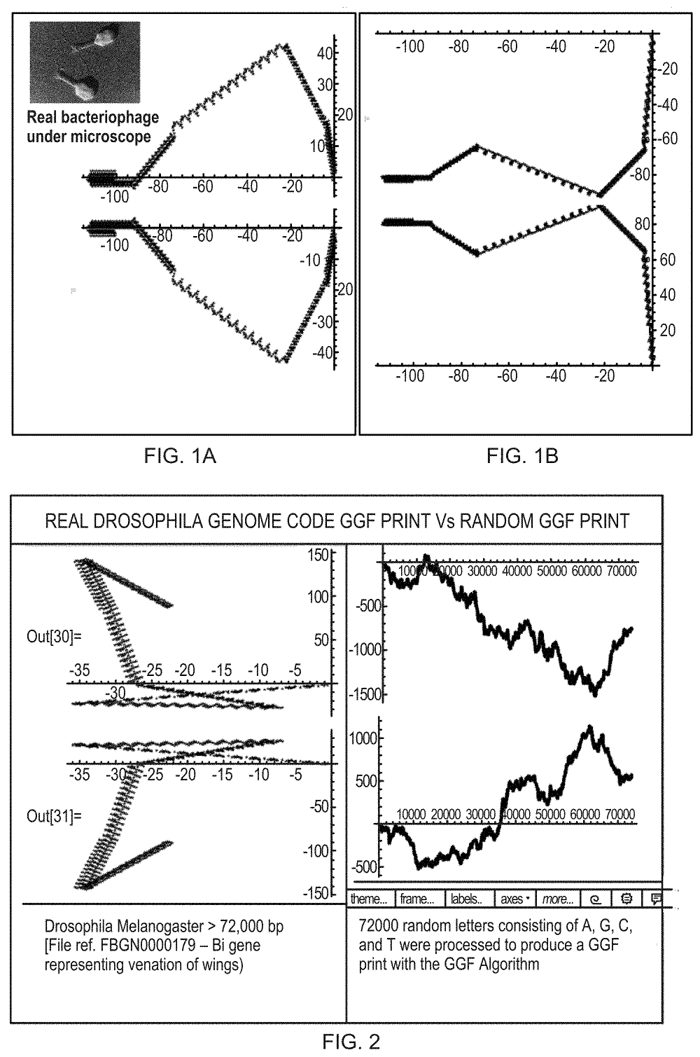

Both the original version and the current version has been applied to the G4 Bacteriophage genome of 5577 bases and when applied the resulting values are mapped to a cartesian graph to produce an image which is considered to resemble the body plan of the bacteriophage (See FIG. 1)

FIG. 1 discloses two GGF print images resulting from application of the GGF algorithm (in Mathematica code) to the 5577 bases of the G4 Bacteriophage. FIG. 1A uses the standard GGF algorithm where the GGF prints symmetry by reversing the order of DNA letters. FIG. 1B is an alternative GGF print involving a different base order to explore whether the phage DNA is capable of expressing different types of phage morphologies.

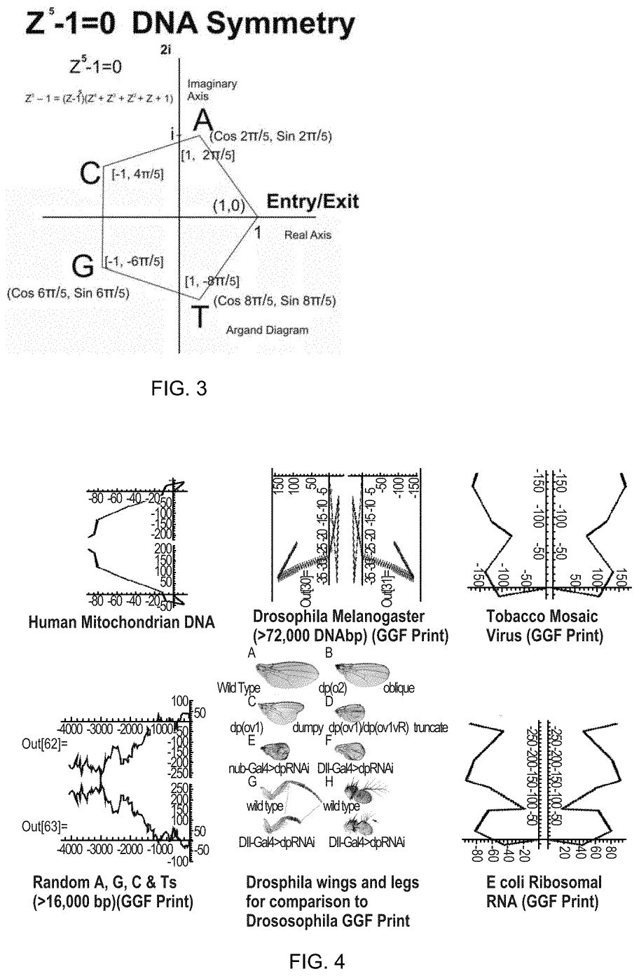

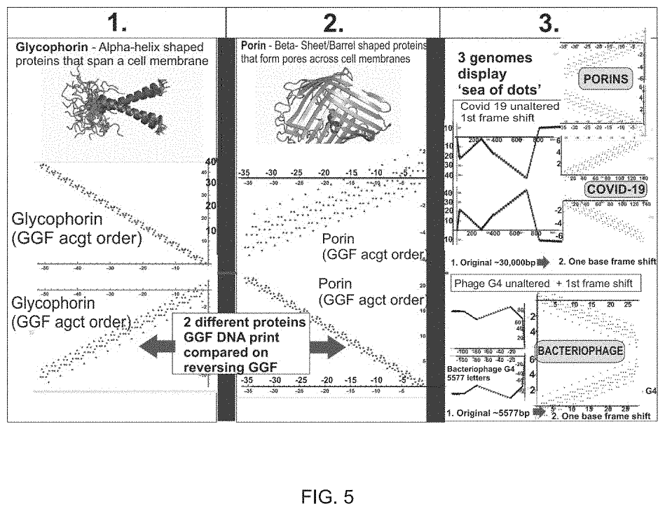

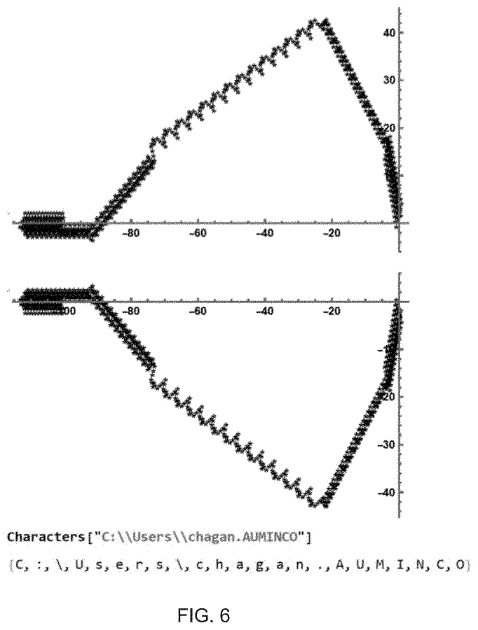

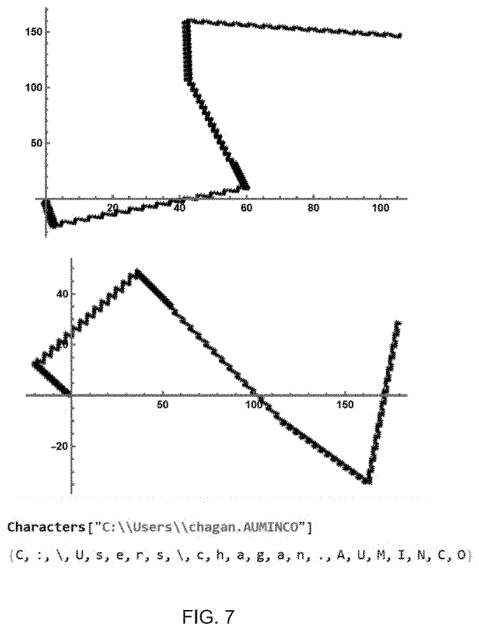

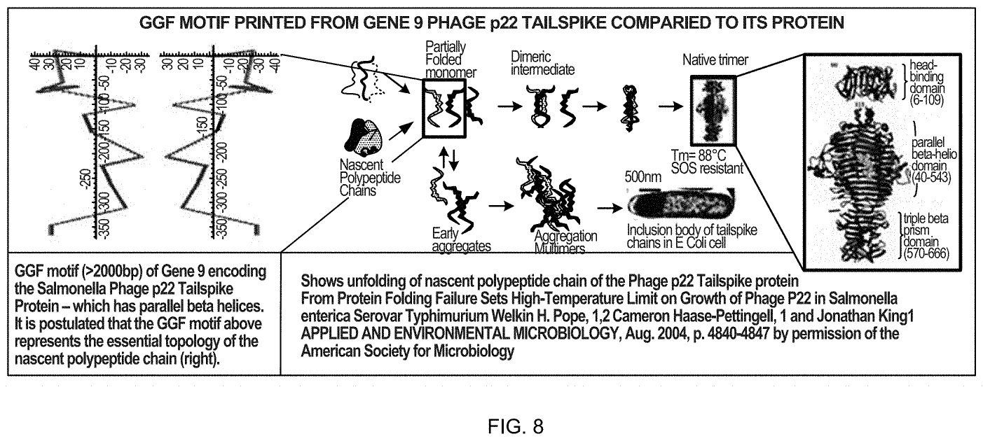

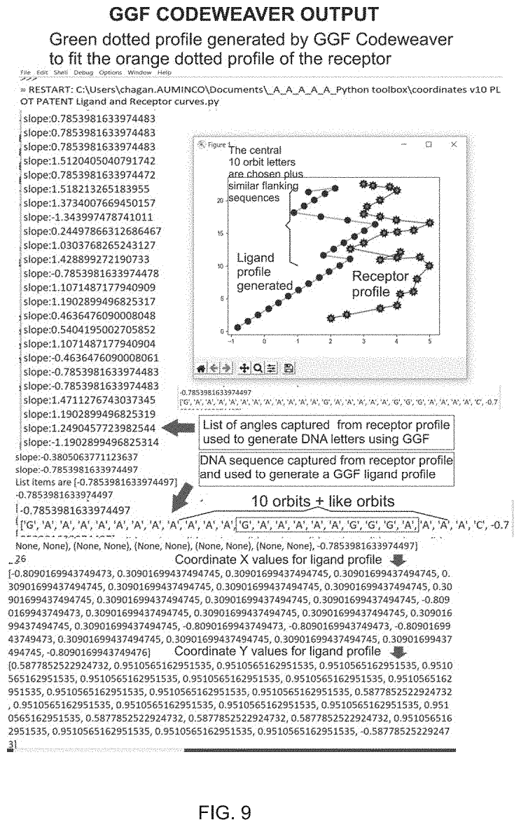

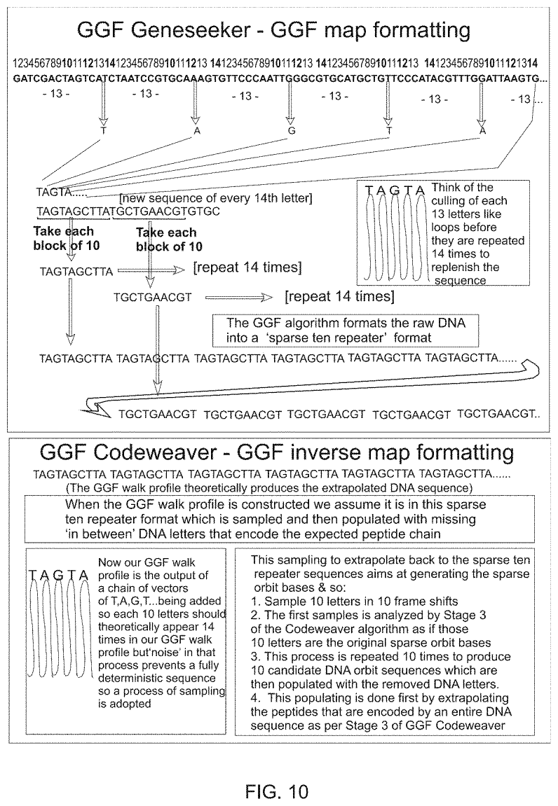

In addition the current version of the GGF algorithm (in Mathematica code) has now been applied to part of the genomes of the Drosophila melanogaster fruit fly, the PAX 6 gene (human eye), mitochondrion (human), 16s sub unit in the ribosomal DNA in the ribosome of E coli, Glycophorin protein, Porin protein, Phage p22 Tailspike protein and all produced ‘body plan’ or structural type images of curved shapes, helices, spirals, patterns etc. In contrast, the GGF algorithm applied to random base sequences produces non-body plan images of ‘gibberish’. For example, a comparison diagram (see FIG. 2) of the GGF algorithm applied to a real 72,000 base sequence from the Drosophila melanogaster Fruit Fly and compared to 72,000 random DNA bases clearly shows that the GGF algorithm is capturing a real meaningful representation on the real Drosophila base sequence Cartesian graph as compared to a non meaningful representation on the Random base sequence Cartesian graph.

The GGF prints of FIG. 2 were conducted to test whether the GGF algorithm images were merely mathematical curiosities (for example, like fractals) or whether a real meaningful function like effect was the product of the DNA code itself. Clearly the 72,000 random letter print is a truly random curve and not smooth, meaningful nor function like. Thus, it suggests the exon—intron stretches are non random as a number of authors suggest and include intergenic regions which are not ‘junk’.

In other words, the algorithm applied to say the 5577 DNA letters of the G4 Bacteriophage produces an image resembling the Bacteriophage body plan despite the GGF program being ‘blind’ as to what shape the Bacteriophage was. The scientific inference is that the mathematical functions inherent in the GGF algorithm program deciphers (to some extent) DNA Code transcription during protein synthesis and how the expression of proteins manifests themselves in their deployment as shapes or patterns of structure (via polypeptides, protein folding or other microcellular synthesis or even macrocellular synthesis) and/or the binary coding of concentration levels of polymers to form the morphology of the body plan of the Bacteriophage (in the sense postulated by Turing and Kauffman's work on morphology).

Now the Bacteriophage is a virus and thus is generally without introns and presumably “100% exons” or all genes. Thus, upon the central hypothesis that the GGF algorithm is able to represent an actual geometric outcome of the DNA code then the image produced by the GGF algorithm represents an abstracted body plan of the bacteriophage synthesized from its genes.

A more complex genome such as Drosophila melanogaster is expected to have exons and introns mixed i.e. ‘genes in pieces’ to use the term used in a paper by biologist Eugene Koonin and others. Curiously, it has been found that the GGF algorithm when applied to such a more complex genome, still produces meaningful curves and shapes reminiscent of a body plan or parts of a body plan. The present inventor and other researchers have hypothesized that the GGF algorithm has decoded a repertoire of potential forms of proteins yet to be synthesized—sequence modules or nascent/candidate genes for nascent/candidate proteins—which form a cascade of pseudogenes. By comparison, researchers such as Ted Steele and his co researchers have found that redundant pseudogenes undergo reverse transcription from RNA to DNA and presumably originally were transcribed from DNA To RNA which together constitutes a recursive feedback process. If this hypothesis is right then it would allow previews of potential genetic processes yet to be manifested in the body (new proteins—irregular proteins—isoforms of proteins) or records of processes that have already taken place (cancer or genetic diseases). The GGF algorithm would thus have both research and clinical use. Again this is mere background postulates of theory which does not detract from the essential utilities of the GGF method over NGS, research and industry.

B.2 GGF Codeweaver

The GGF Codeweaver is applied to the profile of a biomolecule to generate a DNA sequence and/or amino acid sequence encoded or implied by that sequence by creating an optimized parallel profile which can form the basis for the shape or structure of a model designer molecule such as a pharmacophore. The DNA sequence and amino acids are extrapolated by the implied geometric encoding of the biomolecule's profile deciphered according to a series of GGF based algorithms intended to perform an inverse GGF mapping from that profile to a parallel profile encoding partial DNA sequence which is then populated to become a full DNA sequence using implied amino acids and codon- to DNA schemes. Some of the sequences have randomized insertions to both fill unknown segment sequences and generate more candidate sequences which can be virtually screened or subjected to wet lab microarray testing or directed evolution.

Taking a major use of the GGF Codeweaver component of the GGF method, namely, drug design to demonstrate the utility of the GGF inverse component it is useful to take both a general example here and a worked example (in Section C).

Drug design is really the essence of pharmacology and drug design is really ligand design. Note that the body has natural ligands which can act as agonists to switch on a protein function (‘activation’) or antagonists to block that function (‘inhibition’). The core aim is for a designed ligand to bind efficiently to the receptor site which does involve many factors depending on features of both the ligand and receptor. So docking programs such as Autodock subject pharmacophores (either ligands from an existing database eg the protein database or novel ligands created for that purpose) to various analysis to check number of bonds, geometry, energies etc. However, the shape, structure and charge are the key determinants. GGF Geneseeker is designed to predict shape and structure from a known DNA sequence. GGF Codeweaver is designed to predict a DNA sequence (and its encoded amino acids) by curve fitting or shadowing the profile of the receptor or relevant biotic curve. Generally, structure in drug design involves 3D structure while shape can be a profile. However, the GGF Codeweaver can shadow multiple profiles of the receptor profile to extrapolate to 3D if necessary.

To take an example, FIG. 8 sets out the GGF Geneseeker print of the DNA sequence encoding the Gene 9 Phage p22 Tailspike protein. Thus, the applicant believes that the GGF print represents the profile of a strand of molecules from that protein (shown on far right diagram). If a biologist intends to find a ligand to lock onto that protein for a pharmaceutical objective then the biologist must identify a pocket as the receptor for that ligand. For convenience call the profile the ‘seahorse profile’ (as the profile seems to resemble a slender sea horse remembering such a strand may be flexible with multiple conformations). Assume now that the seahorse is hydrophobic and that it and its fellow strands are also hydrophobic. As it nestles in with its fellow seahorse strands in an aqueous medium the water forces those strands to bend inward from the water forming a recess on the protein—a seahorse shaped ‘pocket’. This recess is one receptor that a biologist might target with a designed ligand designed to fit that recess. Now either the biologist can search a database for known ligands of a similar shape, structure and charge that might dock into that recess or it might design a novel ligand from chemicals or biologics. One example of a ligand here might be an aptamer—a type of chemical antibody that is made of DNA or RNA designed to fold up into a ligand that might dock into a receptor. If so the GGF Codeweaver can take the profile of the recess and curve fit to extract a DNA sequence encoding a protein that may fold to fit that recess ie shadow that profile by applying its multiple stage algorithms to extrapolate a DNA or RNA sequence that would fold up into a ligand shaped to dock with the seahorse shaped receptor. If the designed ligand binds efficiently to the receptor then a drug made of these ligands does its job eg it could be designed to kill the phage virus. The binding efficiency of that ligand would first be tested by, most likely, a docking program (eg Autodock) testing how easily the molecular forces assemble the ligand with the receptor to form a complex. Then (or alternatively) the biologist could conduct wet screening tests to see the success rate for how the ligand binds with the receptor (eg the ligand might be fluorescently tagged so that the ‘lit up ligand’ would show on electron microscopy in a microarray).

So just to summarize that example the DNA sequence that might encode a candidate ligand is unknown. The GGF Codeweaver processes the profile of the seahorse pocket to extrapolate a DNA sequence that expresses a protein having a profile of a similar shape or optimized shape that suits docking with the seahorse receptor. That ligand could be an aptamer that folds in the right shape to dock with the receptor on the phage protein or it could be a protein expressed by the DNA sequence that produces a peptide chain that folds into the right shape. GGF Codeweaver can produce multiple different candidate DNA sequences for that purpose. It could also take multiple profiles of the receptor recess at different 3D frames to produce multiple sequence sets and then extract the intersections from sets (ie the DNA sequence segments common to two or more sets).

Note this is only one example of a biological design process where the GGF Codeweaver might be used to aid design. Some of the other processes where GGF might aid the design process include ligands to fit a cancer protein that drives excessive growth or to inhibit a gene expressing itself by targeting the site of the gene in DNA, a ligand oligonucleotide to come between 2 proteins interacting as part of a metabolic pathway where it is sought inhibit or activate that pathway or designing a ligand to activate or inhibit an enzyme for an industrial biosynthetic process. Ligands can mimic natural ligands in the body or can be synthetic made of a fragments of previous ligands which can be assembled partly with other fragments to make up a new ‘mix and match’ ligand made of a scaffold with these fragments. Such fragments can be chemical or biological and thus wherever a DNA or RNA sequence can create a molecule that can contribute to this scaffold the GGF Codeweaver can contribute. Even the final molecular sequence can use molecules substituted for the standard DNA or RNA bases ie instead of A, G, C, T or U which sometimes takes place to optimize or stabilize features of the final drug. For example, aptamers made of DNA or RNA have been found useful to attach to chemical scaffolds to act as delivery vehicles for the core therapeutic molecules making up the drug.

What is the utility of the GGF Codeweaver in the context of the design of biologics, biosimilar and other biosynthetic molecules?

See Annexure A for a table of drugs quoting ligands used where the first drug used a random sequence of amino acids (Glu, Ala, Lys, Tyr). GGF Codeweaver can create an initial pharmacophore eg an aptamer based on a designed sequence of DNA/RNA rather than a random sequence of DNA/RNA or expressed amino acids.

The different techniques and problem solving in drug design is complex but a good overview can be obtained from Drug Design entry in Wikipedia https://en.wikipedia.org/wiki/Drug_design and it can be said generally that the GGF Codeweaver can aid many aspects of drug design particularly the ‘reverse pharmacology’ or ‘indirect drug design’ concepts referred to in that entry (when the shape/structure of the target receptor is available). In contrast when it is not available directed evolution—wet lab micro array techniques would generally be used where GGF Codeweaver would not have direct application with one exception—where homology based target receptors are constructed from which a generic profile might be taken to allow Codeweaver to generate candidate sequences from).

A good indication of cutting edge techniques in the drug design process to create new ie novel drugs is found in a paper on a java based software for ‘evolutionary de novo molecular design’. (See Molegear: A Java-Based Platform for Evolutionary De Novo Molecular Design—Yunhan Chu & Xuezhong He—Molecules 2019, 24, 1444) The authors note that the chemical space for design of such drugs is ‘huge’ and refer to evolutionary algorithms and such docking programs as Autodock which assist the design process. Many papers quote the biologic biochemical space at ˜10¹⁵ due to the vast permutations and combinations of the 4 DNA bases (T equating to U) or the 20 amino acids expressed by DNA. They refer to a known ligand database—the National Cancer Institute—has having 140,000 compounds. The Molegear library itself has 1151 fragments and 552 scaffolds which provide a generic ‘toolbox’ where one can work from known ligand fragments or structures. Again the ‘split and screen’ strategy (mixing and matching fragments) involves exponential increases in potential ligands. They take an example of the HIV drug ie ligand indinavir and use the Molegear software against the target receptor to design a new candidate ligand to compare it to indinavir—a real drug that inhibits the HIV-1 protease. They refer to using 30 generations of directed evolution of target ligands, repeating each type of design 6 times using random numbers and using ‘related fragments’ (presumably fragments resembling parts of indinavir) to arrive at an indinavir like ligand but conclude: “However, this is almost impossible in practice due to a huge combinatorial space”

This underscores the enormity of the task ie even ‘knowing the answer’—the shape of indinavir could not produce the highest score result—at best those 8 indinivir fragment backed candidates came in ‘good’ but were not the best candidates with ‘shape’ being the prime determining criterion. Indinivir, which was on the market in 1996, was designed using molecular modelling and the X-ray crystal structure of the target enzyme complex. The terminal molecules were hydrophobic increasing binding potency (‘charge’). (See the “Discovery and development of HIV-protease inhibitors” Wikipedia entry https://en.wikipedia.org/wiki/Discovery and development of HIV-protease inhibitors).

With the GGF Codeweaver it is envisaged that a biologist, in constructing a receptor profile—call it a Biocurve—would categorize each segment of the molecular pocket profile with being alpha helical, beta sheet like or a turn and also whether hydrophobic or hydrophilic. For example, the biologist might observe antiparallel beta sheets in the Biocurve receptor yet decide to classify that part of shadow curve with parallel beta sheets if the ligand is to be a protein. If the ligand is to be made of DNA as in an aptamer then an antiparallel beta sheet need only bind with the grooves on DNA yet classifying the GGF walk curve assists with populating the polypeptide chain as an intermediate step before extrapolating a full DNA sequence for the aptamer. (See DNA recognition by beta-sheets—M. Tateno et al doi: 10.1002/(SICI)1097-0282(1997)44:4<335). Charge is relevant also. A typical globular molecule might be mostly covered with beta sheets with say one or two alpha helices integrated or as offshoots with hydrophilic molecules on the exterior (due to attraction to water) and the hydrophobic molecules snugly bound in the centre. Thus, in this simple model the biologist might classify a pocket as beta sheets for part, alpha helix for part and all hydrophilic. Yet if the pocket was deep the biologist might elect to nominate part hydrophilic and part hydrophobic. For example, porins proteins which have pores have hydrophobic molecules on the exterior of the pores yet hydrophilic on the inner lining of the pores (permitting the water to channel through them).

Both the identification and design of the Biocurve and its categorization just explained is not a trivial exercise. The biologist might view the different visual representations of the receptor using such methods as space filling, wireframe, ribbon or ball and stick diagrams to discern a Biocurve suitable to extrapolate a shadow GGF walk motif forming the basis for a ligand. The Drug Design entry in Wikipedia (ibid) comments: “A biomolecular target (most commonly a protein or nucleic acid) is a key molecule involved in a particular metabolite or signalling pathway that is associated with a specific disease condition or pathology or to the infectivity or survival of a microbial pathogen.” The Biocurve receptor must be chosen on the theory that the right ligand will modulate the target by inhibiting or activating its function.

A useful paper which demonstrates the task of ‘finding the Biocurve/receptor profile’ involves an unusual technique of inverse pharmacology. In “SynPharm and the guide to pharmacology database: A toolset for conferring drug control on engineered proteins” by Jamie A. Davies November 2020 Protein Science 30(1) doi; 10.1002/pro 3971 the author discusses ligands that can inhibit/activate novel metabolic pathways by identifying a ‘ligand binding domain’ (a receptor) ‘suitable for transfer to effector molecules such as the two CRISPR molecules cas9 and Cpfl. The relevance of the article is not this complex technique of identifying and transferring a receptor ‘module’ to another molecule but that it highlights the task in finding the Biocurve receptor itself (which Codeweaver needs to take a profile from to generate a candidate ligand). Importantly, in identifying the (Biocurve) receptors the author states the criteria for selection of receptors on a target protein as “ . . . the extent to which it is formed from a relatively self-contained run of amino acids, forming a structure relatively independent of the rest of the protein. This ‘ligand-binding module’ can itself be highly folded, as long as it is relatively self contained.” It is the profile of that module that GGF Codeweaver extrapolates a DNA sequence from.

Now set out below an extract from that paper being 2 out of 15 rows of the table of receptors (‘ligand binding domains’) each of which contains details of the receptor pocket eg number of residues (ie the number of amino acids in the peptides forming the folded protein containing the target receptor) and the percentage of the whole protein the pocket represents. For example here are 2 lines from that table:

| | | | | | Proportional |
|---|---|---|---|---|---|
| ID | Target | Species | Ligand | Length | length |
| 84366 | Glucagon | Human | NNC0640 | 59 | 10.1% |
| | receptor | | | | |
| 82905 | MMP1 | Human | CGS-27023A | 61 | 35.7 |

All Drug Responsive Elements which Respond to a Guide to Pharmacology Ligand 644

So the lengths here of 59 to 61 residues would imply say 59 to 61 codons encoded by a DNA sequence of about 180 nucleotides. Thus, about 12 to 13 DNA letters would be extrapolated from a Biocurve implying 180 nucleotides (recalling that the GGF Geneseeker removes 13 nucleotides in between each 14th nucleotide used to plot each pixel on a GGF motif diagram). In Section C we refer to the GGF nucleotides (here each 14^{th} nucleotide) as orbit nucleotides or letters. Thus, if an aptamer is being designed these 12 to 13 nucleotides would have intermediate nucleotides encoding the residues. The GGF Codeweaver algorithms (demonstrated in C below) extrapolate those residues both from the 12 to 13 nucleotides and the classification of the molecules in the Biocurve.

The authors of the Molegear article also comment regarding molecular assembly design diagrams: ‘The graph-based representation has a good resemblance of the constitution of a chemical structure, which can be easily manipulated by human knowledge. It should be noted that the properties of a chemical structure are highly dependent on the 3D structure, and thus an appropriate 3D structure is usually required which is generated by an explicit program such as Balloon in Molegear. Both atoms and fragments can be used as basic building blocks for the assembly of candidate structures.” (under ‘Molecular Assembly’). The authors also discuss molecular features in constructing the model: “A set of molecular descriptors (eg electronic, geometrical, topological and hybrid categories—imported from the CDK QSAR package are used to capture the chemical features of an involved molecule or fragment set” (under ‘Chemical Space Analysis’). They go on to discuss using scoring with these features before plotting the model and comment further; “Regression by means of projections to latent structures (PLS) method, and classification by k-nearest neighbors (K-NN) can be further investigated by QSAR/QSPR analytical tools.”

The Drug Design entry in Wikipedia (ibid) comments on drug design:

“The reality is that present computational methods are imperfect and provide, at best, only qualitatively accurate estimates of affinity. In practice, it takes several iterations of design, synthesis and testing before an optimal drug is discovered. Computational methods have accelerated discovery by reducing the number of iterations required and have provided novel structures.”

The GGF Codeweaver method makes an important contribution to reduce the number of iterations required and also provide novel structures in a very challenging biochemical space where the potential DNA sequences that can produce a ligand are ‘huge’. (quoting the Molegear authors). To take one instance, aptamers are particularly good for cancer drug candidates, use no proteins nor organisms yet generally they use a screening method called SELEX (selection of functional nucleic acids) which uses random sequence oligonucleotides in a typical experiment. A typical pool of oligonucleotides of up to 10¹⁵ members is then subjected to generations of directed evolution to yield candidate aptamers. GGF Codeweaver would start with determined DNA sequences rather than purely random sequences which should speed up the process. One paper quoted 6 days of testing for one evolutionary series of experiments and so shortening these times should not only improve candidate quality but significantly improve success rates due to faster development times.

### C. Specific Description to Enable Someone Expert in the Art to Replicate and Use the Invention

C.1 GGF Geneseeker

In embodiments of the invention, the GGF Geneseeker method including the GGF algorithm the subject of this application processes a formatted sequence of bases in DNA or RNA—optionally DNA—and analogous sequences of other macromolecules such as amino acids in polypeptide chains e.g. proteins. In the preferred embodiment, the bases are ‘code units’ of DNA (or RNA) in the sense used in the central dogma, namely, that the 4 DNA bases, adenine, guanine, cytosine and thymine (or adenine, guanine, cytosine and uracil in the case of RNA) are processed according to their formatted linear order in the DNA double stranded macromolecular chain (or a single stranded chain in the case of RNA). Each base so processed in the formatted order (such formatting including omitted or repeating of bases or both) and by the GGF algorithm is processed in such formatted order in the DNA macromolecule recursively to produce a source dataset containing a series of angles which are representatively set at radial intervals around the origin of a Cartesian graph or Argand diagram according to the chosen angular scheme. This angular scheme involves substituting (‘converting’) each base to each angle according to this portrayal of the scheme:

‘A’, ‘G’, ‘C’, ‘T’ to angles ⊖:

$$A \to \frac{{r}_{1} π}{n} ,$$

$$G \to \frac{{r}_{2} π}{n} ,$$

$$C \to \frac{{r}_{3} π}{n} ,$$

$$T \to \frac{{r}_{4} π}{n}$$

The settings of this scheme of angles occur by modifying the variables r₁, r₂, r₃, r₄ and n and can be set by the researcher as required for the purpose of the analysis to be carried out.

So too, the settings for the formatting of the linear order of bases in DNA can be similarly set. These settings will involve periodical deletion or omission of bases (or both) and corresponding generation of repeater sequences of the remaining bases as will be seen in the example below.

These formatted settings are in their simplest form (without base deletions) to use the raw DNA code order of bases without change to create the source dataset from which bases will be extracted to be converted to angles before mapping/charting onto the Cartesian or other diagram. In one embodiment, a more complex formatting will occur which will involve this procedure:
(i) the removal of a periodic series of bases leaving remaining bases in the source dataset;
(ii) the remaining bases are then repeated a number of times to form a new source dataset which is referred to above as the formatted sequence of bases in DNA or RNA;

The starting position is any starting position in the relevant DNA sequence in the source dataset and (a ‘GGF reading frame’) then that frame can be moved one base at a time to create a series of GGF motifs for further study. Further variations of formatting can be chosen by the user.

So in detail this is how a researcher would use the GGF method:

A. The user may utilize computer software capable of coding algorithms and interfacing with databases storing a biological data set.

B. The user would load the standard code (or could construct its own bespoke code with selected angle and formatting settings) into its computer running such software. For example,

Attachment A has a full printout of the Mathematica code for this embodiment that is representative of the code that would be loaded into the user's computer to enable the GGF formatting Algorithm to work.

C. The user would need to prepare a dataset of a chain of macromolecules containing a postulated code which the researcher wishes to analyse or catalog into a database or library and such dataset needs to have the raw code sequence of molecules e.g. DNA bases into the dataset optionally without spaces between each letter representing a molecule (base) and designate this dataset with a file name (call it DSFILENAME)

D. The user would enter DSFILENAME into the GGF code that it has loaded into its computer.

E. The user would then execute the program to run the code which accesses the code sequence in DSFILENAME.

F. A graph is then produced which will generally display a pattern of dots forming a distinctive curve which is designated the GGF motif.

G. The image of the GGF motif is then analysed and can then be stored in a relational database or library for future reference or analysis.

H. The researcher can change the reading frame or the settings or both and can then repeat the procedure to run executions of code and then compare the resulting GGF motifs that are generated and these can also be stored in the database for future reference.

Examples of GGF motifs resulting from execution of the Mathematica code in Attachment A can be seen also in Attachment A. The reading frame and formatting settings in the GGF example in Attachment A are indicative of how a researcher might change future settings. Thus, taking this example of the GGF print for the Bacteriophage G4 the following settings apply and guidance added:

(a) the reading frame starts with the first base on an altered Bacteriophage genome. This alteration is done by removing the first base and commencing the GGF on the second base causing a GGF frame shift of one base which can produce interesting results—for example in FIG. 5 the ‘sea of dots’ common to both Bacteriophage and Covid-19 viruses resulted from a GGF frame shift of one base (i.e. commencing GGF on the second base). A researcher can experiment with any number of frame shifts to suit his or her purposes;

(b) the ‘framewrap’ formatting settings in attachment A involve removing the first 13 bases (i.e. the 1^{st} to 13^{th} bases) leaving the 14^{th} base and then removing the next 13 bases and keeping the next 14^{th} base and so on. The ‘repeater’ formatting settings involve just repeating the remaining 14^{th} bases 10 times to amplify the source dataset. Now it is thought that these 2 formatting settings approximate how genes can be accessed by transcription factors according to an epigenetic code system. For example, DNA is wrapped around the nucleosome core particles in about 147 bp per nucleosome. (see Munshi Al, Shafi G, Aliya N, Jyothy A. Histone modifications dictate specific biological readouts. J Genet Genomics. 2009 February; 36(2):75-88. doi: 10.1016/S1673-8527(08)60094-6.). In addition, the geometry of DNA involves icosahedral and pentagonal symmetry (top down view of helix) where 10 pentagons are nested around each 2π rotation of the helix. (See Vanessa Hill Peter Rowlands, Nature's code, October 2008 DOI: 10.1063/1.3020651). Thus, about each 14^{th} base could be an access point for transcription. So too repeater sequences of 10 could merely be a mechanism for tiling of sub units in the body eg tiling of proteins in cellular coating which might be modelled on Penrose Tiling.

(c) In the same way the angle settings of the GGF algorithm can be varied according to the particular geometrical approach the researcher adopts e.g. with Attachment A the GGF angle settings are called ‘pentagonal GGF’ settings (described below). One postulated outcome could be that such settings produce pentagonal tiling in cellular coating because pentagons do not tile in a plane and more typically tile in a 3D surface such as the surface of a truncated icosahedron (triangles and pentagonal tiling) and thus more likely to tile cell surfaces (as to the protein coating of the Bacteriophage G4 it has an icosahedral head without pentagons but could be postulated as a pre-truncated icosahedral phase or alternatively a pentagonal tiling process to produce triangular sub units noting that DNA exhibits both icosahedral and pentagonal symmetries).

Thus, each researcher can adopt or change these settings to conform with his or her particular biological doctrine, hypothesis or theory that they wish to apply to their work when using the GGF method.

An inspection of the Mathematica Code in Attachment A will enable any user to see what settings for {r₁, r₂, r₃, r₄} and n have been used and in addition the base deletions and repeater settings used in that code. The user can thus vary these settings as required.

C.2 GGF Codeweaver

The GGF Codeweaver method comprises 4 stages which includes algorithms (attached) is described as follows:

Stage 1. GGF Walk curve—the front end of the GGF Codeweaver method involves generating a curve to shadow a biotic profile on a cartesian x-y plane similar to a random walk except the ‘walk’ (called the GGF walk) is not random. The GGF walk is created by taking a profile of a biotic form (the Biocurve) such as a cancer protein to be targeted for drug treatment (the ‘receptor’) or the receptor could be an antigen eg. the spike on the surface of a virus is targeted as a docking site for a drug or vaccine. (For convenience we will refer to a ‘receptor’ below and the drug to dock with it as the ‘ligand’ but a vaccine docking with an antigen could equally be used with the method) or a biosynthetic enzyme designed to activate or inhibit a biological pathway in an industrial biochemical medium. The walk can be fully predetermined by the Biocurve or be partly pre determined and partly random.

The aim is to direct the GGF walk to shadow the path of the Biocurve as closely as possible so the GGF walk becomes a jagged shadow of the Biocurve. In this embodiment of the invention, the GGF walk is then converted to a DNA sequence encoding a candidate ligand that would fit that receptor as part of a drug design process to produce a pharmacophore. The conversion from that ‘GGF shadow’ of the Biocurve occurs by choice of a procedure to reverse the GGF algorithm—the generalized inverse mapping of the GGF algorithm.

The GGF walk is thus an extrapolated GGF motif that is predicted to have been generated by the GGF algorithm of an unknown DNA sequence encoding that GGF motif (Recall that DNA sequences appear to produce GGF motifs representing the morphology of the organism or molecular sub-unit encoded by that DNA sequence.) Thus, the GGF walk represents the theoretical mapping of the GGF algorithm applied to an unknown DNA sequence and the extrapolation of that GGF curve back to a predicted DNA sequence via an approximated inverse mapping of the GGF algorithm.

Recall also that the GGF algorithm proceeds via a ‘step by step’ walk governed by the conversion of each base in the DNA sequence to an angle producing an angled vector that predetermines the chain of vectors ie the steps making up a determined GGF motif.

The GGF walk is not a predetermined GGF motif as the DNA sequence is unknown.

Rather the GGF walk is generated by producing a chain of vectors—steps ‘hugging’ the Biocurve ie approximating its profile step by step—each step being one of the 4 angled vectors. A preferable method to generate a profile that will fit the Biocurve is to capture a discretized list of angles of the discretized profile eg the biologist converts the biotic profile of the receptor target to a list of 2D coordinates. The lines between each 2 points can then be viewed a chain of vectors whereby the ‘chain of angles’ of these vectors can also be captured. This chain of angles is then processed by one preferred version of the GGF Codeweaver method to produce a profile of a candidate ligand that can fit the target receptor whose profile is shadowed by the Codeweaver profile. The coordinates of the Biocurve can be discretized ideally as one unit per nucleotide because the shadow GGF walk is intended to extrapolate an orbit nucleotide on a one for one basis—eg if the Biocurve is 150 nucleotides in length this implies say 150/14 orbit letters ie ˜11 orbit letters. However, sometimes it may be difficult to construct a precise one unit per nucleotide coordinates for reasons of complexity or convenience. In such a case GGF Codeweaver can optimize a shadow GGF walk curve from such a Biocurve that is not uniformly discretized on the one unit per nucleotide basis. In fact the example in Annexure B has a Biocurve that is not so discretized. This may even reflect reality due to the flexibility in conformation of both the ligand and receptor eg bonds may be rotatable etc. If there is not a one for one correspondence ie not one unit per nucleotide, then the issue of granularity of the Biocurve arises. The biologist should frame the Biocurve such that each shift in angle greater than 2Pi/5 around say 2 or 3 coordinates equals one nucleotide in the shadow curve. Thus in this case the Biocurve might need to be stretched or shrunk or if the Biocurve is taken ‘as is’ the granularity can be scaled within the GGF Codeweaver algorithm if need be—eg each unit of angle change could be enlarged or shrunk via a scale setting (this is not presented in the example in Section C).

An example of this preferred procedure is set out as Python Code in Annexure B with its output diagram shown in Annexure C. This procedure is deterministic.

An alternative procedure which can be wholly or partly deterministic (ie it can be partly random) is for each step to be chosen by ‘reaching’ out with all 4 possible vectors and then choosing the vector that reaches a point nearest to the Biocurve. Alternative procedures for generating the chain of vectors can be chosen to widen the pool of candidate ligands or pharmacophores. In detail this stage 1 of the GGF Codeweaver proceeds as follows:
(a) A list of coordinates is produced representing the Biocurve. The working coordinates are merely the (x,y) coordinates on a cartesian graph but the coordinates will have an additional ‘hidden non 3D’ z coordinate encoding each xy coordinate as one of 3 different types of segments categorized by the biologist. This hidden z coordinate is not needed for this Stage 1 of the project and will be referred to again in Stage 2 and thus can be ignored for the method in Stage 1;
(b) In the algorithm a code line becomes the ‘walk engine’ to generate steps eg in Python the Min (minimum value from a list) can be used to choose the slopes/gradients of the GGF walk (ligand) to be closest to the receptor Biocurve (receptor) slopes/gradients. Alternate methods in Mathematica might use Nestlist or Foldlist functions which can combine deterministic and randomized algorithms in the method eg if this latter method is followed in Mathematica then prior to each step by the ‘walk engine’ the reaching of 4 moves being the 4 angled vectors to test each move—the correct move—the vector that falls closest to the Biocurve becomes the move for that step;

Stage 2. Input data formatting The biocurve formatting of coordinates—this is done as (x,y,z) coordinates—not 3D—the z is the classification of coordinates into alpha helices, beta sheets or turns by the biologist. The idea is that the biologist will have a receptor in mind and wishes to develop a novel ligand (or hybrid of novel fragments mixed with known fragments). The biologist will take the most promising profiles of the receptor eg a profile containing a cleft or groove—a pocket for the ligand. The Biocurve must be taken at a compatible granularity which has been explained earlier. Once this Biocurve profile is taken the biologist will have to design guidelines in mind eg Lipinski's rule of 5 where molecular weight of less than 500 daltons, no more than 5 hydrogen bond donors, no more than 10 hydrogen bond receptors etc are ‘favourite’ features. In visualizing this ‘drug like’ molecule we would ask the biologist to suggest whether the new ligand (whose profile is the Biocurve) would be alpha helix or beta sheets or turns at its various segments. Recall earlier the example of 180 nucleotides implying 12-13 orbit nucleotides and intermediate nucleotides in a 180 nucleotide aptamer. Later we use a scheme based on a paper ‘Nature's Code’ where each orbit nucleotide is treated as the central letter of a peptide triplet. Thus, 12-13 nucleotides become 12-13 orbit based peptides (ie 12-13 amino acids).

Thus, if there were say 10 orbit based peptides of the Biocurve represented by 10 coordinates then these might be (x,y) pairs of:

{1,2},{3,4},{5,5},{8,7},{8,7},{9,8},{10,10},{10,11},{11,13},{12,14} which when the z coordinate representing an alpha helix (A), beta sheet (B) or turns (T) is added might appear as follows:

### {1,2,B},{3,4,B},{5,5,B},{8,7,B},{8,7,B},{9,8,T},{10,10,A},{10,11,A},{11,13,A},{12,14,A}

This is the Biocurve which is then approximated by the GGF walk shadow curve which will present a list of coordinates of about the same size—probably not the same number of coordinates. Then these coordinates are formatted similarly as (x,y,z) co ordinates by adding (non 3D) z coordinates that match the Biocurve z coordinates as closely as possible.

Stage 3. Protein Sequence Generation: The protein sequence generation starts by taking the GGF walk curve of (x,y,z) coordinates just referred to and then converting these into polar coordinates {r₁,θ₁}, {r₂,θ₂}, . . . {rₙ,θₙ} from each {x,y,_} of the {x,y,z} coordinates. A list with only the angle θₙ from each co ordinate is constructed ie

{θ₁,θ₂, . . . θₙ}

Then using the reverse of the GGF algorithm each angle θₙ is converted to a DNA letter. Recall that the DNA letters that are plotted in GGF motifs are not the original DNA sequence. This is obtained by removing 13 out of 14 of the original letters to create a ‘sparse’ DNA sequence of orbit letters which was 1/14^{th} as long as the raw DNA sequence of say 150 nucleotides. Then every 10 letters of this new sparse sequence is multiplied by 14 to replenish that list to about the same length as the raw list. (See FIG. 9 in the schedule to this description for a fuller explanation). So the list of angles are converted to:

{g,a, . . . t} or {g,a t,c,g,a,t . . . }

Our research shows that this formatting of settings at removing 13 from 14 can be varied with similar results indicating that the DNA code overlaps in its expression functions. This is fortunate because it turns out that the 13 base removal every 14 bases is more difficult to populate systematically because if operative overlapping codons exist then the complexity rises exponentially. The solution is to adjust the setting to avoid overlapping codons and this can be achieved by choosing say removing 14 out every 15 bases with each remaining 15^{th} base being orbit letters. The theory is that the GGF algorithm reflects the wrapping of DNA in nucleosomes in orbits which are accessed through ‘gene silencing’ events during gene expression with the orbit letters perhaps being cornerstones for loops of DNA/RNA in protein synthesis. Thus if each 15^{th} letter is treated as the orbit letter this involves 14 letters theoretically missing from our initial capture of ‘sparse’ orbit letters from the Biocurve. How do we find the missing intermediate letters in this sparse sequence? First, we would treat each orbit letters as a central letter and convert it to a triplet via a scheme detailed below. This reduces the unknown intermediate letters from 14 to 12 letters. This then leaves 12 letters each representing 4 codon triplets which encode the peptide chain represented by the profile of the Biocurve (ie the profile of the protein receptor we are targeting). Secondly, we then use biochemistry using structure, charge and other chemical features to predict the most likely amino acids making up that peptide chain. In short, alpha helices, beta sheets or turns, hydrophobic or hydrophilic (polar or non polar), steric compatibility using volumes (size of molecules) noting that many features overlap or are neutral between the major categories—a true exercise in combinatorial complexity where pragmatism must temper too much precision.

So in reversing our GGF walk profile (the model GGF motif we are trying to reverse map to the original DNA sequence we must reflect this reverse mapping by building in this formatting in reverse which is achieved by selecting the best fitting 10 of the captured DNA letters from the GGF walk generated by Stage 1 above (see FIG. 9 which explains the rationale for this sampling). So taking these 10 orbit letters can be captured as a sequence of ‘sparse’ DNA letters (remember 14 letters would have theoretically been removed between each of them if we were conducting the forward GGF instead of the reverse GGF inverse)—in FIG. 9 the example given was TAGTACGTTA (SEQ ID NO: 7). On the list below they are first separated by 14 blank spaces with the 2 blank spaces flanking each orbit letter converted into to form peptide triplets using a scheme involving certain triplets being favoured when the central letter from the Nature's Code scheme triplets matches the respective orbit letter. (The user can select any scheme known or unknown to biology. Here we adopt a scheme suggested by a paper “Nature's Code” Vanessa Hill Peter Rowlands, Nature's code, October 2008 DOI: 10.1063/1.3020651—referred to as “the Nature's Code Scheme”)

An example is worked through below adopting the Nature's Code scheme to populate the flanking missing letters to each orbit letter to form triplets. (The Nature's Code scheme sees the central letters as a crucial criterion in the DNA code and suggests classifying features of amino acids encoded by triplet codons according to their central letters). We then designate the resulting triplet temporarily as Os for clarity (each O is a triplet) before converting them into 8 types consisting of 2 categories of the 4 types of Os linking to each letter that generated the central orbit letter in the triplet ie each central letter in an O triplet links to one of A, G, C or T and we use the 2 hydropathic categories of each triplet ie hydrophobic or hydrophilic.

To take an example this could produce a sequence of orbit letters like this:

[____G____A____T____C____G____A____T . . . ]

The orbit letters are then assumed to be the central letter of triplet codons suggested by the Nature's Code scheme and so we then use the biologists categorization of Biocurve to categorize the matching GGF walk shadow curve. (These categories include alpha helix (+,−), beta sheets (+,−) or turns explained later). The categories ‘force’ choices from each respective codon group, first, by matching codons that have the same central letter (eg agc and aga are from the same codon group suggested by the Nature's Code scheme) and second by matching codons with greater affinity to their respective category. So the sequence should now look like:

[_____AGC_____CAT_____GTC_____TCT_____AGA_____
GAG_____CTA . . . ]

Note there only 4 types ie _A_, _G_, _C_ & _T_ shown here but each of the 4 types is either hydrophilic (likes water call it +) or hydrophobic (does not like water call it −) so we will adopt 8 types _A_+, _G_+, _C_+, _T_+, _A_−, _G_−, _C_−, _T_−

Just to keep these 8 orbit letters earmarked temporarily so we can start populating the intermediate triplet (peptides) assume all eight categories are just O (because O is only an intermediate symbol before it converts to 8 different symbols) and so we now have:

[____O____O____O____O____O____O____O . . . ]

[Note: here show 7 ‘O’ peptide triplets from the 10 peptides forming part of 50 peptides or 150 nucleotides]

Each O is a codon triplet—meaning that there are 12 blank spaces theoretically left between each O—recall that 14 blank spaces were left between orbit letters before orbit letters were expanded into triplets. Now although there are 12 spaces these translate only 4 triplets (amino acids) forming part of the peptide chain and as these amino acids have the chemical affinities (matching the biologists categories) they allow us to populate the 12 spaces or 4 codon slots encoding 4 amino acids using those features.

Those 4 intermediate amino acids (12 letters) are unknown but we now assume that the biologist has given the designation of z in the coordinates of the Biocurve segmented profile which tells us whether they are alpha helix (A) beta sheet (B) or turns (T). To be more specific the biologist further classifies these into 5 types; Alpha Helix(hydrophilic)−A, Alpha Helix (hydrophobic)—a, Beta Sheet (hydrophilic)—B, Beta Sheet(hydrophobic), turns (T).

Earlier we discussed the granularity and discretization regarding one to one correspondence between nucleotides on the Biocurve and extrapolated nucleotides on the GGF walk shadow curve.

In Annexure C we saw that the central 10 orbit letters we obtained from processing the Biocurve (receptor) were:

| (SEQ ID NO: 8) | |
|---|---|
| | GAAAAAGGGA |

If, for a particular biological rationale, the biologist wished to decrease the granularity subjectively then a wider segment of orbit letters to base our ligand could be captured. (Alternatively, granularity might be adjusted by varying the algorithm settings). Here instead of letters: ie GAAAAAGGGA (SEQ ID NO: 8), the biologist might widen the selection to 15 letters which we now assume for the rest of this example:

(SEQ ID NO: 9)
AAAGAAAAAGGGAAA

These need to become:

OOOOOOOOOOOOOOO
O____O____O____O____O____O____O____O____O____
O____O____O____O____O____O____O]

And now we need to find out the full amino acid sequence that makes up the Codeweaver Ligand curve that Stage 1 has captured. In doing this recall the biologist has classified the segments of the curve (ie the candidate ligand) as follows (orbit triplets are bold ‘O’ ie each ‘O’=_o_

### Codeweaver Ligand Curve Amino Acid/Peptide Chain

[‘O’,‘A’,‘A’,‘A’,‘b’,‘O’,‘b’,‘b’,‘A’,‘A’,‘O’,‘A’,‘A’,‘t’,‘B’,‘O’,‘B’,‘B’,‘B’,‘B’,‘O’,‘A’,‘A’, ‘A’,‘t’,‘O’,‘B’,‘B’,‘B’,‘B’,‘O’,‘a’,‘a’,‘a’,‘a’,‘O’,‘a’,‘t’,‘A’,‘A’,‘O’,‘A’,‘A’,‘A’,‘b’,‘O’,‘b’,‘b’,‘A’,‘A’,‘O’,‘A’, ‘A’,‘t’,‘B’,‘O’,‘B’,‘B’,‘B’,‘B’,‘O’,‘A’,‘A’,‘A’,‘t’,‘O’,‘B’,‘B’,‘B’,‘B’,‘O’,‘a’,‘a’,‘a’,‘a’]

### Legend:

O=an orbit triplet whose central letter is an orbit base

A=triplet prefers alpha helix that is hydrophilic,

B=triplet prefers beta sheet that is hydrophilic,

a=triplet prefers alpha helix that is hydrophobic,

b=triplet prefers beta sheet that is hydrophobic,

t=turn no preferred hydropathy

The scheme for populating the Codeweaver Ligand curve that has been adopted is reflected in the Python code set out in FIG. 9 but we will first explain how that scheme is constructed (although there are extensive comments within that code—comments are preceded by # for each comment line).

Now we are in a position to populate the missing flanking bases to each orbit letter inside each O using the Nature's Code scheme (which is similar to existing schemes anyway—note that all codes show that any amino acid scheme is a combinatorial dilemma as to allocating features to groups to codons.) In the present example feature groups have been made based on discretion which might vary from biologist to biologist. The essential method remains the same whichever feature scheme is chosen.

The next step is to take the intermediate triplets ([‘O’,‘A’,‘A’,‘A’,‘b’,‘O’,‘b’,‘b’,‘A’ . . . these are the underlined ones in the above Codeweaver Ligand Curve amino acid/peptide chain where we can see that the biologist has designated 5 types (see under Legend above). Now to take an instance the biologist might consider that the target protein receptor has hydrophilic molecules on the outside and hydrophobic molecules on the inside or sees a porin type molecule where hydrophilic molecules appear on the inner pores of the protein. In any event the biologist first considers whether structurally an alpha helical ligand or a beta sheet ligand (or mixture) is best and then designates them hydrophilic (A,B) or hydrophobic (a,b).

Then each letter A, B, a, b, t is replaced by random choice from ‘jars’ which is achieved in the Python Code in Annexure B by putting the amino acids whose properties match the A, B, a, b, t categories into ‘jars’. The metaphor is choosing ‘lollies’ (amino acids) from a jar. (A biologist can vary the allocation of amino acids in jars depending on which allocation scheme they support.) These jars are lists of amino acids classified into various names which refer to the features of the categories of amino acids/codons remaining (jar names are JarA—Alpha helix (+), JarB—Beta Sheet (+), Jara—alpha helix (−), Jarb—beta sheet (−) and so Jart on—5 jars) Other jars are then created for the orbit letters (explained below) or to minimize steric clashes eg JarAr for lower volume alpha helix (+) to be re selected during the program to reduce steric clashes. How do we allocate the amino acids to the respective jars earmarked to replace the triplet letter ([‘O’, ‘A’,‘A’,‘A’,‘b’,‘O’,‘b’,‘b’,‘A’ . . . ?

First, the propensity of amino acids to form Alpha Helices, Beta Sheets or Turns is statistically known—see table of Berg, Tymoczko & Stryer—Biochemistry 5^{th} Edition Berg, Tymoczko & Stryer—Biochemistry 5^{th} Edition, W H Freeman & Co. New York p67—table of relative frequencies of amino acid residues. Second, Nature's Code mentions classifications for the hydrophilic vs hydrophobic criterion. Thus 2 criteria allow us to allocate the correct amino acids into the correct jars which can then be used to replace each triplet ([‘O’,‘A’,‘A’,‘A’,‘b’,‘O’, ‘b’,‘b’,‘A’ . . . accordingly (where there is choice left and no third criteria the Python code chooses randomly.

Now how do we replace the O orbit triplets with the correct amino acids? Each O triplet is flanked by an alpha (+,−), beta (+,−) or turn and we know the central letter of each triplet. Thus, from the 20 amino acids consisting of 20 triplets (some with varying third ‘wobble’ bases not relevant here) we need to convert each orbit O to an amino acid.

Here we also have 2 criteria to use to select amino acids encoded by those triplets. An important context which governs Stage 3 of the Codeweaver method under the Nature's Own scheme is qualified by the table shown in FIG. 10. For example, in the table F refers to Phenylalanine which can be constructed via the codons TTT or TTC. P or Proline has CCU, CCC, CCA and CCG. So we can see the first criteria of the central letter reducing the 20 choices down to 5 choices (20/4=5). The second criteria of alpha (+,−), beta (+,−) or turn cannot be discerned from the table in FIG. 10 but ‘matching’ the flanking amino acid classification reduces the choices down again to a fairly narrow band. Again this is achieved in the Python Code in Annexure C by putting this narrow band of remaining choices again into ‘jars’ which are classified into various names which refer to the features of the choices (the amino acids/codon) remaining (these jar names such as JarOTP—hydrophilic triplets with central letter T, JarOCP—hydrophilic triplets with central letter C, JarOAp—hydrophobic triplets with central letter T, and so on (8 jars).

Stage 4 extrapolating a DNA sequence from the amino acid sequence: The amino acid—DNA codon table in FIG. 10 now guides the design of ‘jars’ in the Stage 4 algorithm set out in Annexure E. Each amino acid is replaced by a random choice of codon triplet governed by that codon table. Note that the central letter of each group of codons in each jar has the same central letter which is important because orbit triplet amino acids started with orbit letters captured from the Biocurve and these are preserved. The ‘contamination by non orbit triplets’ during shuffling in Stage 3 (to minimize steric clashes by volume comparisons) was avoided by keeping orbit triplets as numerals (representing volumes). Thus, the Stage 4 algorithm converts both the ‘letter’ amino acid and the ‘numeral’ amino acid orbit triplets to a random choice of DNA codons to generate the final DNA sequence. Thus, the output of the Stage 4 algorithm in Annexure E is shown in Annexure F.

To explain the 4 stages in Annexures, the following table of stages is shown:

| | Stage | Output |
|---|---|---|
| A | Stage 1 | Stage 1 |
| | The Biocurve of the receptor (or | (These coordinates appear at the bottom of FIG. 9 |
| | antigen) Is shadowed by the GGF | and are indicated as a list of X values and a |
| | Walk represented by X, Y | separate list of Y values (separated for convenience |
| | coordinates which is generated by | in the Python code) |
| | the Stage 1 algorithm eg such as | |
| | is contained in the Python code in | |
| | Annexure B | |
| B. | The XY coordinates of the GGF | (These angles appear at the bottom of Annexure C |
| | walk shown in Annexure C | and are encoded as slope 0, slope 1, slope 3 etc. |
| | display vector angles common to | These angles then convert to: |
| | each X & Y coordinate and these | AAAGAAAAAGGGAAA (SEQ ID NO: 9) |
| | angles are then converted to DNA | |
| | base letters | |
| C | Stage 2 is formatting input for | Stage 2 |
| | Stage 3 using Stage 1 output ie | AAAGAAAAAGGGAAA (SEQ ID NO: 9) |
| | Stage 2 is not an algorithm but the | Is subjected to formatting by the biologist |
| | task of a biologist classifying the | |
| | GGF Walk coordinates ‘tagged’ | |
| | with a third Z coordinate for | |
| | additional features Alpha | |
| | helix (+) Alpha Helix (−), Beta | |
| | Sheet (+), Beta Sheet (−) or Turn | |
| D | (Stage 2 cont.) Add ‘Z’ coordinate | AAAGAAAAAGGGAAA (SEQ ID NO: 9) |
| | to each base (being converted to | formatted to allow flanking bases: |
| | triplets) to classify them into | _A_A_A_G_A_A_A_A_A_G_G_G_A_A_A |
| | Alpha helix+[A] Alpha Helix-[a], | And then classified into |
| | Beta Sheet +[B], Beta Sheet-[b] or | bAb_AAA_BAB_BGA_tAB_Baa_aAa_aAA_AA |
| | Turn[t] | A_bGb_AGA_BGB_BAA_tAA_BAa |
| E | (Stage 2 cont.) For simplicity of | bAb_AAA_BAB_BGA_tAB_Baa_aAa_aAA_AA |
| | explanation here we temporarily | A_bGb_AGA_BGB_BAA_tAA_Baa |
| | define each triplet here as a triplet | Define bAb, AAA, . . . etc = O, O, O . . . etc |
| | O to help explain the next step | becomes: |
| | where: | O_O_O_O_O_O_O_O_O_O_O_O_O_O_O or |
| | O = an orbit triplet whose central | O____O____O____O____O____O____ |
| | letter is an orbit base | O____O____O____O____O____ |
| | | O____O____O____O |
| F | (Stage 2 cont.) The intermediate | Then this sequence of Os becomes populated with |
| | 12 bases connecting each orbit | the Biologist's classification of the GGF walk |
| | triplet would also have been | curve: |
| | tagged/classified by the Biologist | [‘O’, ‘A’, ‘A’, ‘A’,‘b’, ‘O’, ‘b’, ‘b’, ‘A’, ‘A’, |
| | and these 12 unknown bases are | ‘O’, ‘A’, ‘A’, ‘t’, ‘B’, ‘O’, ‘B’, ‘B’, ‘B’, ‘B’, |
| | converted to unknown triplets via | ‘O’, ‘A’, ‘A’, ‘A’, ‘t’, ‘O’, ‘B’, ‘B’, ‘B’, ‘B’, |
| | the same tagging scheme ie | ‘O’, ‘a’, ‘a’, ‘a’, ‘a’, ‘O’, ‘a’, ‘t’, ‘A’, ‘A’, |
| | Alpha helix+[A] Alpha Helix- | ‘O’, ‘A’, ‘A’, ‘A’, ‘b’, ‘O’, ‘b’, ‘b’, 'A’, ‘A’, |
| | [a], Beta Sheet +[B], Beta Sheet- | 'O’, ‘A’, ‘A’, ‘t’, ‘B’, ‘O’, ‘B’, ‘B’, ‘B’, ‘B’, |
| | [b] or Turn[t] | ‘O’, ‘A’, ‘A’, ‘A’, ‘t’, ‘O’, ‘B’, ‘B’, ‘B’, ‘B’, |
| | | ‘O’, ‘a’, ‘a’, ‘a’, ‘a’] |
| G | (Stage 2 cont.) Each O base is to | ‘J’, ‘A’, ‘A’, ‘A’, ‘b’, ‘J’, ‘b’, ‘b’, ‘A’, ‘A’, |
| | be re classified using the same | ‘J’, ‘A’, ‘A’, ‘t’, ‘B’, ‘Z’, ‘B’, ‘B’, ‘B’, ‘B’, |
| | scheme(note 0 # O as O is a base | ‘J’, ‘A’, ‘A’, ‘A’, ‘t’, ‘j’, ‘B’, ‘B’, ‘B’, ‘B’, |
| | and O is a triplet): | ‘j’, ‘a’, ‘a’, ‘a’, ‘a’, ‘j’, ‘a’, ‘t’, ‘A’, ‘A’, |
| | J = a hydrophilic triplet that has an | ‘J’, ‘A’, ‘A’, ‘A’, ‘b’, ‘z’, ‘b’, ‘b’, ‘A’, ‘A’, |
| | orbit base ‘A’ as its central letter, | ‘Z’, ‘A’, ‘A’, ‘t’, ‘B’, ‘Z’, ‘B’, ‘B’, ‘B’, ‘B’, |
| | Z = a h'philic triplet that has an | ‘J’, ‘A’, ‘A’, ‘A’, ‘t’, ‘a’, ‘J’, ‘B’, ‘B’, ‘B’, |
| | orbit base ‘G’ as its central letter | ‘B’, ‘j’, ‘a’, ‘a’, ‘a’, ‘a’ |
| | X = a hydrophilic triplet that has | |
| | an orbit base ‘C’ as its central | |
| | letter, | |
| | U = a h'philic triplet that has an | |
| | orbit base ‘T’ as its central letter | |
| | j = a hydrophobic triplet that has | |
| | an orbit base ‘a’ as its central | |
| | letter, | |
| | z = a h'phobic triplet that has an | |
| | orbit base ‘g’ as its central letter | |
| | x = a hydrophobic triplet that has | |
| | an orbit base ‘c’ as its central | |
| | letter, | |
| | u = a h'phobic triplet that has an | |
| | orbit base ‘t’ as its central letter | |
| H | Stage 3 input completed: | Stage 3 |
| | Stage 2 is shown as the completed | ‘J’, ‘A’, ‘A’, ‘A’, ‘b’, ‘J’, ‘b’, ‘b’, ‘A’, ‘A’, |
| | input sequence of orbit triplets | ‘J’, ‘A’, ‘A’, ‘t’, ‘B’, ‘Z’, ‘B’, ‘B’, ‘B’, ‘B’, |
| | and intermediate triplets | ‘J’, ‘A’, ‘A’, ‘A’, ‘t’, ‘j’, ‘B’, ‘B’, ‘B’, ‘B’, |
| | (‘J’, ‘A’ . . . ) which are then | ‘j’, ‘a’, ‘a’, ‘a’, ‘a’, ‘j’, ‘a’, ‘t’, ‘A’, ‘A’, |
| | inputted into the Codeweaver | ‘J’, ‘A’, ‘A’, ‘A’, ‘b’, ‘z’, ‘b’, ‘b’, ‘A’, ‘A’, |
| | Stage 3 program set out in Python | ‘Z’, ‘A’, ‘A’, ‘t’, ‘B’, ‘Z’, ‘B’, ‘B’, ‘B’, ‘B’, |
| | Code in Annexure C with output | ‘J’, ‘A’, ‘A’, ‘A’, ‘t’, ‘a’, ‘J’, ‘B’, ‘B’, ‘B’, |
| | shown in Annexure D | ‘B’, ‘j’, ‘a’, ‘a’, ‘a’, ‘a’ |
| | | Is converted by the Stage 3 code in Annexure C |
| | | to Annexure D namely the Amino Acid sequence |
| | | below which forms the basis for the ligand: |
| | | [114, ‘D’, ‘R’, ‘E’, ‘V’, 138, ‘T’, ‘T’, ‘E’, ‘E’, |
| | | 114, ‘R’, ‘R’, ‘D’, ‘D’, 173, ‘T’, ‘R’, ‘E’, ‘R’, |
| | | 138, ‘E’, ‘E’, ‘K’, ‘P’, 153, ‘S’, ‘L’, ‘N’, ‘T’, |
| | | 193, ‘C’, ‘F’, ‘C’, ‘F’, 193, ‘A’, ‘P’, ‘R’, ‘E’, |
| | | 193, ‘R’, ‘E’, ‘D’, ‘T’, 227, ‘L’, ‘V’, ‘R’, ‘R’, |
| | | 60, ‘D’, ‘D’, ‘N’, ‘E’, 173, ‘G’, ‘S’, ‘P’, ‘R’, |
| | | 111, ‘D’, ‘E’, ‘R’, ‘D’, ‘F’, 193, ‘P’, ‘S’, ‘T’, |
| | | ‘W’, 153, ‘C’, ‘C’, ‘C’, ‘a’] |
| | | [numbers can be converted to amino acids where |
| | | required but here they serve only as intermediate |
| | | values to be replaced by codons in Stage 4] |
| I | Stage 4 | Stage 4 |
| | The sequence of amino Acids | [‘AAC’, ‘GAT’, ‘AGG’, ‘GAA’, ‘GTG’, ‘GAA’, |
| | outputted by Codeweaver Stage 3 | ‘ACT’, ‘ACA’, ‘GAG’, ‘GAA’, ‘AAT’, ‘AGA’, |
| | are then processed by the | ‘AGG’, ‘GAT’, ‘GAC’, ‘AGA’, ‘ACG’, ‘AGA’, |
| | Codeweaver Stage 4 program set | ‘GAA’, ‘AGA’, ‘GAG’, ‘GAA’, ‘GAG’, ‘AAA’, |
| | out in Python Code in Annexure E | ‘CCG’, ‘CAC’, ‘TCA’, ‘TTA’, ‘AAT’, ‘ACC’, |
| | with output shown in Annexure F | ‘TAC’, ‘TGC’, ‘TTC’, ‘TGC’, ‘TTC’, ‘TAT’, |
| | | ‘GCC’, ‘CCC’, ‘AGG’, ‘GAG’, ‘TAT’, ‘AGA’, |
| | | ‘GAG’, ‘GAT’, ‘ACA’, ‘TGG’, ‘TTA’, ‘GTT’, |
| | | ‘AGA’, ‘AGA’, ‘GGC’, ‘GAT’, ‘GAC’, ‘AAT’, |
| | | ‘GAG’, ‘AGG’, ‘GGA’, ‘TCT’, ‘CCA’, ‘AGG’, |
| | | ‘GAC’, ‘GAC’, ‘GAA’, ‘AGG’, ‘GAC’, ‘TTT’, |
| | | ‘TAT’, ‘CCC’, ‘TCT’, ‘ACG’, ‘TGG’, ‘CAC’, |
| | | ‘TGT’, ‘TGC’, ‘TGT’, ‘a’] |

The DNA sequence in the last stage 4 shown in the table above then forms the basis for a de novo biologic DNA sequence which must then be synthesized and then subjected to small sample randomized clinical trials to generate valuable data typical of larger trials. This can be virtual screening via docking, QSAR or other programs or can proceed to wet lab screening using microarrays of receptors to test binding success rates or combined with a directed evolution program such as development of aptamer drug candidates. The sequence might also be used as a ‘fragment’ and combined with existing known scaffolds. If the DNA sequence produces a prospective ligand or vaccine then it can be considered for drug development with testing on mice before consideration of human trials. Once appropriate patient populations can be ascertained Phase 1 trials could begin.

### D. Mathematical Description of the Invention to Explain the Principles in Formulating the GGF Algorithm and Formatting Code Embodied in the Sample Mathematica Code—Attachment A

The background mathematical specifications which may be required for a biomathematician assisting the researcher is now set out below.

An example of the GGF algorithm is if n=5 and {r₁, r₂, r₃, r₄}˜2, 4, 6, 8} then this produces what is referred to as a pentagonal GGF (see FIG. 3) because the co ordinates produced by the mapped angles (shown below) represent a pentagon on an Argand diagram which are the roots of unity of the equation, Z⁵−1=0 which means that the solutions are 5 points being co ordinates representing 4 complex solutions and one real solution:

$$A \to \frac{2 π}{5} \qquad Pentagonal GGF$$

$$C \to \frac{4 π}{5}$$

$$G \to \frac{6 π}{5}$$

$$T \to \frac{8 π}{5}$$

A second example (amongst many forms) of the GGF algorithm is if n=17 and {r₁, r₂, r₃, r₄}˜{8, 16, 24, 32} then this produces what is referred to as a Heptadecagonal GGF because the co ordinates produced by the mapped angles represent a Heptadecagon on an Argand diagram which are the roots of unity of the equation, Z¹⁷−1=0 which means that the solutions are 16 points being co ordinates representing 16 complex solutions and one real solution but unlike the Pentagonal GGF, only some of the complex solutions are chosen to fit r₁, r₂, r₃, r₄:

(Note the best configuration of angles to exhibit DNA symmetry was chosen for the heptadecagonal GGF.)

See Attachment B for a full printout including GGF Motif of the Mathematica code for this embodiment of the GGF Algorithm at work.

r₁, r₂, r₃, r₄ were selected to exhibit symmetry between pairs of A, C, G or T according to base pairing or non base pairing coupling.

However, the embodiment of the GGF algorithm can have r₁, r₂, r₃, r₄ and n selected as desired in simple or more complex linear applications or functions as the case may be. The preferred mathematical form of the GGF Algorithm appears below as G(F(x))=(a·Cos[c·F(x)], b·Sin[d·F(x)]) which is a parametrization then inserted into a recursive function H(x).

$$H ( x ) = ( {\sum }_{i = 1}^{i = k} a . Cos [ c . F ( {x}_{i} ) ] , {\sum }_{i = 1}^{i = k} b . Sin [ d . F ( {x}_{i} ) ] )$$

H(x) creates a vector path such that each angle is treated as a vector and added recursively and cumulatively to each prior angle vector (each outcome of G(F(x)) where x is each base drawn in a queue in base order from the source dataset to produce the co ordinates that make the GGF Motif on a Cartesian graph (or Argand diagram). However, alternative functions are further forms of the GGF algorithm which can be used depending on the requirement and would replace the above general form of co ordinates and become (U(F(x), V(F(x)) where U or V can be trigonometric or other functions. One example, of U and V could be (SinxCosx, CosxSin2x) or many other variations. Kaufman and Turing used different Sine functions to model differentiation and morphology and thus varying the GGF algorithm is a useful research technique.

FIG. 3 shows a selection of {r₁, r₂, r₃, r₄}˜{2, 4, 6, 8} in the GGF Algorithm that produces what is referred to as a pentagonal GGF because the co ordinates produced by the mapped angles represent a pentagon on an Argand diagram where the 5 points of the pentagon are the roots of unity of the equation, Z⁵−1=0.

Note that G & C have been exchanged relative to A and T in many GGF demonstration prints and can be one of the many user setting changes depending on configuration of GGF algorithm chosen.

In populating the source dataset, the recursive linear processing converts each base to such an angle according to the particular scheme to match the bases to angles. Thus the first step in this processing taken by the algorithm converts the series of bases to a series of angles to fill the source dataset. The second step is that preferably these angles are then mapped and converted to Cartesian Co ordinates using polar co ordinates or imaginary co ordinates on an Argand diagram. The simplest form of this step would be to take the series of angles θ1, θ2, θ3 . . . . θn to coordinates (cos θ1, sin θ1), (cos θ1+cos θ2, sin θ1+sin θ2), (cos θ1+cos θ2+cos θ3, sin θ1+sin θ2+sin θ3), . . . (Σᵢ₌₁ⁿ cos θn, Σᵢ₌₁ⁿ sin θn).

The third step is to then chart these co ordinates from the source dataset onto a Cartesian graph or Argand diagram to create an image or curve or curves which we refer to as the GGF Motif.

In a more complex form the mapping of angles would, instead of mapping to a simple series (cos θ1, sin θ1), . . . Σᵢ₌₁ⁿ cos θn, Σᵢ₌₁ⁿ sin θn) be a more complex (a cos [cθ1], b sin [dθ1]), . . . (Σᵢ₌₁ⁿ a cos [cθn], Σᵢ₌₁ⁿ b sin [dθn]) or other possible parametrizations.

In yet an even more complex form the mapping could involve each angle being parametrized so that each angle is mapped by the Cosine or Sine of the image* (ie value) of a more complex function F to its pixel image* (ie coordinate) producing a similarly recursive cumulative series of coordinates (a cos [c.F(θ1)], b sin [d.F(θ1)]), . . . (Σᵢ₌₁ⁿ a cos c.F(θn}], Σᵢ₌₁ⁿ b sin [dF(θn)]). (*the first use of ‘image’ is in the mathematical sense ie f(x) is the image of x under f whereas the second use of ‘image’ is in the graphical sense). However, another yet more complex embodiment could instead map the angles to spherical or cylindrical co ordinates or yet other manifold systems by schemes which can be graded to produce simpler series to more complex series in a similar manner by addition of constants and functions as required.

The above processing, mapping and charting to produce GGF motifs can be done manually, by computer or partly manually and partly by computer using software written in a suitable language to code the algorithm for efficient execution when applied to the series of bases in the dataset being processed to produce such GGF Motifs.

The present working version of the GGF algorithm has been written in Mathematica but could be written in other computer languages if required. The algorithm converts the linear sequence/chain of bases/molecules into a series of Cartesian co ordinates preferably in polar format which are then mapped to become an image called a “GGF Motif” which is postulated to represent a visual representation of molecular structure in a topological and/or morphological sense but may represent other mathematical features or relations yet to be discovered.

An example of one embodiment of the GGF Algorithm is the working of the pentagonal GGF Algorithm (i.e. where {r₁, r₂, r₃, r₄} are selected as ˜{2, 4, 6, 8} and n=5) as applied to the G4 Bacteriophage virus genome and set out below:

1. The 5577 DNA letters of the G4 Bacteriophage were inputted into the source dataset for the ‘G4 Phage Program’.

2. A sequence of letters was then extracted being every 14^{th} letter to form a list of 398 letters out of the 5577 letters to begin formatting the source dataset.

3. Then each block of 10 letters from the 398 letters is expanded to form 14 identical blocks of 10 letters totaling 140 letters per new block (i.e. repeater sequences).

For example, the first new block takes the first 10 letters of the 398 letters and repeats a further 13 times to form the 14 new sub blocks of 10 letters. This expands the 398 to 5460 letters to complete the source dataset formatting. (This means that 39 blocks of 10 letters are expanded 14 times to 5,460 letters with 8 letters remaining as redundant.)

4. Thus the 398 letters have now expanded to about 5,460 letters to form the formatted source dataset.

5. Each letter is then converted into an angle by this transformation: “A”, “G”, “C”, “T” to angles θ: A->2π/5, G->4π/5, C->6π/5, T->8π/5 (the “Conversion Rules”) before Steps E and F listed earlier are performed ie the user then executes the GGF program which recursively and cumulatively creates a series of co ordinates which then adds and plots each resulting vector (each vector serially connecting each two sequential sets of co ordinates) such vector plot then producing a GGF Motif.

In this embodiment of the GGF algorithm they appear in the order A, G, C, T and as will be seen in Part 2 this reflects the fact that A pairs with T and G pairs with C. The Conversion Rules carry out the rotating mappings of bases to angles in the Mathematica code can be chosen by the user. For example, a user might choose A (purine), G (purine) C (pyrimidine) and T(pyrimidine) because ascending angles as A and G are purine (A->2π/5,G->4π/5) and C and T are pyrimidine:

### (C->6π/5, T->8 π/5) in FIG. 3.

As the GGF is cyclic the choice of cycle becomes either Purine(A), Purine (G), Pyrimidine (C), Pyrimidine (T) OR Purine(A), Pyrimidine (C), Purine (G), Pyrimidine (T) which a user might consider more feasible also in a cyclic function. A GGF print using order “A”, “G”, “C”, “T” (Order 1—FIG. 1B) can thus be compared to a GGF print with order “A”, “C”, “G”, “T” (Order 2—FIG. 1A), producing similar mathematical equations and results yet displays how reversal of symmetry (“G”, “C”, to “C”, “G”,) may be a mechanism in morphology. Note that this is not mere palindromic DNA symmetry because GGF generates a recursive ‘function of a function’ output plotting such output as a series of vectors. However, some GGF prints display perfect symmetry by a mere exchange of bases in the GGF (e.g. “A”, →“T” becomes “T”, →“A” in the GGF Mathematica program). Note the Mathematica Program in Attachment A has been reproduced with the Conversion Rules using Order 1 (mod 2π) ascending angles for “A”, “C”, “G”, “T” namely: “A”, “C”, “G”, “T” to angles θ: A->2π/5, G->4π/5, C->6π/5, T->8π/5 (A comparison of GGF prints using Conversion Rules of both Order 1 and Order 2 appears in FIG. 1 above— FIG. 1A vs FIG. 1B.)

The resulting angles from the Conversion Rules are then used to create the cumulative recursive co ordinates (cos θₖ, sin θₖ)(k=1 to k=n) for each letter so that the 5,460 ‘base letters’ become 5,460 angles and finally 5,460 sets of Cartesian Co ordinates joined and plotted as cumulative vectors. (Note (a cos c.θ, b sin d.θ) with a=b=c=d=1 or (r cos θ, r sin θ) with r=1 are just the polar version of (x,y) coordinates.)

6. These Cartesian Co ordinates are then plotted by way of a dot or pixel being placed at each point and this produces an image of the body plan of the Bacteriophage.

A mathematical definition of the GGF Geneseeker algorithm is:

$$Generative Genome Function : G ( F ( x ) )$$

$$F ( x ) = [ \begin{matrix}\frac{2 π}{5} & if x is A \\ \frac{4 π}{5} & if x is C \\ \frac{6 π}{5} & if x is G \\ \frac{8 π}{5} & if x is T\end{matrix} ]$$

$$G ( F ( x ) ) = ( a . Cos c . F ( x ) , b . Sin d . F ( x ) )$$

$$a , b , c and d are constants and have all been assumed to equal 1 in GGF prints$$

And as stated earlier, G(F(x))=(a.Cos[c.F(x)], b.Sin[d.F(x)]) is a parametrization which is then inserted into a recursive function H(x) where each base letter is added to produce each sequential set of coordinates to be graphically joined as a cumulative vector plot.

$$H ( x ) = ( {\sum }_{i = 1}^{i = k} a . Cos [ c . F ( {x}_{i} ) ] , {\sum }_{i = 1}^{i = k} b . Sin [ d . F ( {x}_{i} ) ] )$$

H(x) creates the co ordinate pairs for a vector path such that each sequential pair of co ordinates produced is treated as a vector and added recursively and cumulatively to each prior angle vector (each vector pair outcome of G(F(x)) where x is each base drawn from a ‘base queue’ in base code order from the source dataset to produce the co ordinates that make joined vectors that are the GGF Motif on a Cartesian graph (or Argand diagram).

### Annexures

An example of this method has been written as 3 ‘modules’ of Python Code which are Annexures B, F and H.

This code has had a list of coordinates to simulate a typical profile of a receptor to show pockets or clefts that might appear on a cancer protein or other biotic form. The list of x y coordinates are denoted as lists of X1 and Y1 in that program (the x-y pairs are separated into 2 lists) that is we start with these coordinates of the biotic profile:

[(2, 2), (2.5, 2.5), (3.5, 3.5), (4, 4), (4.1, 5.7), (4.5, 6.1), (4.6, 8), (5, 10), (4.7, 11.3), (3.5, 11), (4.1, 12), (4, 11.3), (2.6, 12.7), (3, 13.5), (3.6, 15), (4, 15.2), (4.5, 15.5), (5, 16.5), (4, 17), (3.5, 17.5), (3, 18), (3.1, 19), (3.5, 20), (4, 21.5), (3.8, 22), (3.3, 22.2), (3, 22.5)]

We insert those coordinates into the Python Code in Annexure C in this form (these entries are seen there)

X1=[2,2.5,3.5,4,4.1,4.5,4.6,5,4.7,3.5,4.1,4,2.6,3,3.6,4,4.5,5,4,3.5,3,3.1,3.5,4,3.8,3.3,3]

Y1=[2,2.5,3.5,4,5.7,6.1,8,10,11.3,11,12,11.3,12.7,13.5,15,15.2,15.5,16.5,17,17.5,18,19,20,21.5,22,22.2,22.5]

Note that later a third z coordinate will be added as explained above.

Z1=([‘A’,‘A’,‘A’‘A’,‘t’,‘B’,‘B’,‘B’,‘A’,‘A’,‘A’,])

Now the Python program extracts the angles from those coordinates of the Biocurve for the receptor and creates a shadow profile for the ligand (the GGF walk shadow curve). The output diagram from this process is shown in FIG. 9 where the green dotted and profile indicate the ligand and the orange dotted profile indicate the receptor.

### Annexure A: Table of Drugs Using Ligand-Receptors

Peptide drugs with high global sales involving peptide ligands targeting key proteins (note the first drug used a random sequence of amino acids— GGF Codeweaver would produce a DNA sequence from a target protein's pocket profile that is not random but which reflects the shape/structure of that profile.

From Chapter—Protein-Peptide Interactions Revolutionize Drug Development In book: Binding Protein by

Berna Sariyar Sariyar Akbulut—DOI: 10.5772/48418

| | Brand | | Target protein/ | |
|---|---|---|---|---|
| Peptide | name | Target disease | biological action | Sequence |
| glatiramer | Copaxone, | Multiple | Unknown | Random mixture of Glu, Ala, |
| acetate | copolymer1 | sclerosis | | Lys, Tyr |
| leuprolide | lupron | Prostate cancer, | Binds gonadotropin- | Pyr-HWSY-D-LLRP-NHEt |
| acetate | | breast cancer | releasing hormone | (SEQ ID NO: 10) |
| | | | receptor | |
| goserelin | Zoladex | Prostate cancer, | luteinising-hormone | p-EHWSY-D-S(tBu)-LRP- |
| acetate | | breast cancer | releasing hormone | AzaGly-NH2 (SEQ ID NO: 11), |
| | | | analog | |
| octreotide | Sandostatin | carcinoid | | H-D-F-c[CFD-WKTC]- |
| acetate | | Acromegaly, | | thiolacetate (SEQ ID NO: 12) |
| | | syndrome | | |
| exenatide | Byetta | Type 2 diabetes | glucagon-like | HGEGTFTSDLSKQMEEEAV |
| | | mellitus | peptide 1 analog | RLFIEWLKNGGPSSGAPPPS |
| | | | | (SEQ ID NO: 13) |
| teriparatide | Forteo | Osteoporosis | | SVSEIQLMHNLGKHLNSME |
| | | | | RVEWLRKKLQDVHNF (SEQ |
| | | | | ID NO: 14) |
| enfuvirtide | Fuzeon | HIV | Targets HIV-1 | Ac-YTSLIHSLIEESQ |
| | | | fusion machinery | QQELNEQELLELD KWASLWNW F-NH₂ |
| | | | | (SEQ ID NO: 15) |

Annexure B: Python Program to Generate a Ligand Profile from a Receptor Profile (Stage 1)
(#=comments)
import math
import numpy as np
import matplotlib.pyplot as plt
import random
from itertools import accumulate
import operator
#CODEWEAVER STAGE 1 PYTHON CODE
#the initial biocurve from a biologist should arrive as a list of ‘2D/3 value’ coords (x,y,z}
#but the z value is not the z axis—it will be the category the co ordinate is eg alpha, beta etc
#Thus we receive the biocurve in the form ((x1,y1,z1), (x2,y2,z2) . . . ) and then we convert this
#into 3 lists of X1=(x1,x2 . . . ), Y1=(y1,y2 . . . ), Z1=(z1.z2 . . . ) to process as set out below:
X1=[2,2.5,3.5,4,4.1,4.5,4.6,5,4.7,3.5,4.1,4,2.6,3,3.6,4,4.5,5,4,3.5,3,3.1,3.5,4,3.8,3.3,3]
Y1=[2,2.5,3.5,4,5.7,6.1,8,10,11.3,11,12,11.3,12.7,13.5,15,15.2,15.5,16.5,17,17.5,18,19,20,21.5,22,22.2,22.5]
#Z1=np.array([‘A’,‘A’,‘A’‘A’,‘t’,‘B’,‘B’,‘B’,‘A’,‘A’,‘A’,])
pi=math.pi
#this iterates through list X and Y and puts the slope of each line between successive points
#into a new list s1 which becomes a list of slopes to print out
for i in range(len(X1)−1) and range(len(Y1)−1):
   s=math.atan(((Y1[i+1]−Y1[i]))/((X1[i+1]−X1[i])))
   print(f“slope:{s}”)
s1=[math.atan(((Y1[i+1]−Y1[i]))/((X1[i+1]−X1[i])))]
print(f“List items are {s1}”)
#this does the same thing except it just puts them into s2 so the length of s1 can be #printed
#this code can be probably made redundant
s2=int(len(X1)−1)
for i in range(int(len(X1)−1)) and range(int(len(Y1)−1)):
   item=math.atan(((Y1[i+1]−Y1[i]))/((X1[i+1]−X1[i])))
   s1.append(item)
#This creates 4 lists
AngleA=[ ]
AngleG=[ ]
AngleC=[ ]
AngleT=[ ]
#These 4 empty lists are then populated with the angles between each successive line on Biocurve
U1=[ ]
W1=[ ]
for i in range(len(s1)−1):
   AngleA=(2*pi/5−s1[i])/(1+s1[i]*2*pi/5)
   AngleG=(4*pi/5−s1[i])/(1+s1[i]*4*pi/5)
   AngleC=(6*pi/5−s1[i])/(1+s1 [i]*6*pi/5)
   AngleT=(8*pi/5−s1[i])/(1+s1[i]*8*pi/5)
   Angles=(AngleA,AngleG,AngleC,AngleT)
   if min(Angles)==AngleA:
      s1[i]=‘A’ and U1.append(math.cos(2*pi/5)), W1.append(math.sin(2*pi/5))
   elif min(Angles)==AngleG:
      s1[i]=‘G’ and U1.append(math.cos(4*pi/5)), W1.append(math.sin(4*pi/5))
   elif min(Angles)==AngleC:
      s1[i]=‘C’ and U1.append(math.cos(6*pi/5)), W1.append(math.sin(6*pi/5))
   elif min(Angles)==AngleT:
      s1[i]=‘T’ and U1.append(math.cos(8*pi/5)), W1.append(math.sin(8*pi/5))
#Now s1 is a list of As,Gs,Cs and Ts which are converted to
#2 lists using U1=Cos(2π*pi/5), W1=Sin(2n*pi/5) which
#combine to become the coordinates for the GGF walk (ligand profile)
GGFDict={“A”: 2*pi/5,“G”: 4*pi/5,“C”: 6*pi/5,“T”: 8*pi/5}
U1P=list(accumulate(U1, operator.add))
W1P=list(accumulate(W1, operator.add))
fig, ax=plt.subplots( )
plt.style.use(‘seaborn’)
ax.scatter(X1,Y1,s=100)
#ax.plot(plt, linewidth=3)
#plt.show( )
plt.scatter(X1, Y1,s=100,)
plt.scatter(U1P, W1P,s=100,)
plt.plot(X1, Y1)
plt.plot(U1P,W1P)
plt.show( )
print(s)
print(s1)
print(s2)
print(U1)
print(W1)

### Annexure C:

### Amino Acids—DNA Codon Table

The Jar references refer to groups of codons for each Amino Acid which the Python program in Codeweaver Stage 4 uses to randomly replace each amino acid in the peptide chain extrapolated in Codeweaver Stage 3 for a codon to create a DNA sequence for a biologic drug or vaccine (note that Stage 3 contains differently named ‘Jar’ groups of symbols for Amino Acids which randomly replace each symbol created by the Pentgrid generated from Stage 1 and Stage 2

| | | | DNA codons matching |
|---|---|---|---|
| Amino Acid | Symbol | Jar | Amino Acid |
| Glycine | G | jarG | ‘GGT’, ‘GGC’, ‘GGA’, ‘GGG’ |
| Alanine | A | jarA | ‘GCT’, ‘GCC’, ‘GCA’, ‘GCG’ |
| Serine | S | jarS | ‘TCT’, ‘TCC’, ‘TCA’, ‘TCG’ |
| Cysteine | C | jarC | ‘TGT’, ‘TGC’ |
| Aspartate | D | jarD | ‘GAT’, ‘GAC’ |
| Proline | P | jarP | ‘CCT’, ‘CCC’, ‘CCA’, ‘CCG’ |
| Asparagine | N | jarN | ‘AAT’, ‘AAC’ |
| Threonine | T | jarT | ‘ACT’, ‘ACC’, ‘ACA’, ‘ACG’ |
| Glutamate | E | jarE | ‘GAA’, ‘GAG’ |
| Valine | V | jarV | ‘GTT’, ‘GTC’, ‘GTA’, ‘GTG’ |
| Glutamine | Q | jarQ | ‘CAA’, ‘CAG’ |
| Histidine | H | jarH | ‘CAT’CAC’ |
| Methionine | M | jarM | ‘ATG’ |
| Isoleucine | I | jarI | ‘ATT’, ‘ATC’, ‘ATA’ |
| Leucine | L | jarL | ‘TTA’, ‘TTG’, ‘CTT’, ‘CTC’, |
| | | | ‘CTA’, ‘CTG’ |
| Lysine | K | jarK | ‘AAA’, ‘AAG’ |
| Arginine | R | jarR | ‘AGA’, ‘AGG’ |
| Phenylalanine | F | jarF | ‘TTT’, ‘TTC’ |
| Tyrosine | Y | jarY | ‘TAT’, ‘TAC’ |
| Tryptophan | W | jarW | ‘TGG’ |

Annexure D: Python Program to Convert Formatted Orbit Base Letters into Triplets Converting into an Amino Acid Sequence to Encode the Ligand (Stage 3)
(#=comments)
#CODEWEAVER STAGE 3 PYTHON CODE
import random
#the scheme is A=triplet prefers alpha helix that is hydrophilic, B=triplet prefers beta sheet that is hydrophilic,
#a=triplet prefers alpha helix that is hydrophobic, b=triplet prefers beta sheet that is hydrophobic,
#t=turn no preferred hydropathy
#J=a hydrophilic triplet that has an orbit base ‘A’ as its central letter, Z=a h'philic triplet that has an orbit base ‘G’ as its central letter
#X=a hydrophilic triplet that has an orbit base ‘C’ as its central letter, U=a h'philic triplet that has an orbit base ‘T’ as its central letter
#j=a hydrophobic triplet that has an orbit base ‘a’ as its central letter, z=a h'phobic triplet that has an orbit base ‘g’ as its central letter
#x=a hydrophobic triplet that has an orbit base ‘c’ as its central letter, u=a h'phobic triplet that has an orbit base ‘t’ as its central letter
#this is a list of captured triplets from a pentgrid graph—each letter is a triplet codon
#Note that orbit letters are O(_A_),Z(_G_),X(_C_),U(_T_) and each
#these represents 3 bases ie a codon—during this program we keep them as codons and fill in the codons between the ‘orbit codons’—these
#‘in between’ codons are coded into A's/a's (alpha helices), B's/b's(beta sheets) & t's—a biologist might trace the profile line on the x-y graph
#as pink (Aa), dark pink (Bb) & grey(t) and these colours become a third coord on the x-y graph ie (x,y,z)—not 3D but only to divide the curve
#into 3 categories so these ‘in between’ codons are then populated—so to take the pentGrid1 list below in between the V,Z,X & T orbit letters we
#have A,a,B,b,t filling those ‘in between’ codons—the task of this algorithm is to convert each letter representing a codon into an amino acid
#in summary the sequence of letters in pentgrid1 are a series of 37 codons—which represent 3×37=111 bases—we don't need to convert to bases yet
pentGrid1=[‘J’,‘A’,‘A’,‘A’,‘b’,‘J’,‘b’,‘b’,‘A’,‘A’,‘J’,‘A’,‘A’,‘t’,‘B’,‘Z’,‘B’,‘B’,‘B’,‘B’,‘J’,‘A’,‘A’, ‘A’,‘t’,‘j’,‘B’,‘B’,‘B’,‘B’,‘j’,‘a’,‘a’,‘a’,‘a’,‘j’,‘a’,‘t’,‘A’,‘A’,‘J’,‘A’,‘A’,‘A’,‘b’,‘z’,‘b’,‘b’,‘A’,‘A’,‘Z’,‘A’,‘A’,‘t’,‘B’,‘Z’,‘B’,‘B’,‘B’,‘B’,‘J’,‘A’,‘A’,‘A’,‘t’,‘a’,‘J’,‘B’,‘B’,‘B’,‘B’,‘j’,‘a’,‘a’,‘a’,‘a’]
#amino acids volumes in A\hat{ }3—these link to the triplets in between the orbit letters (ie orbit bases)
G=60.1
A=88.6
S=89.1
C=108.5
D=111.1
P=112.7
N=114.1
T=116.1
E=138.4
V=140.1
Q=143.8
H=153.2
M=162.9
I=166.6
L=166.7
K=168.6
R=173.4
F=189.9
Y=193.6
W=227.8
#The amino acid volumes below are repeated but rounded so the computer recognizes them as different
#because each base links to a jar in the orbit triplets
g=60
a=88
s=89
c=108
d=111
p=112
n=114
t=116
e=138
v=140
q=143
h=153
m=162
i=166
l=167
k=168
r=173
f=189
y=193
w=227
#AAs for ‘in between’ letters are placed in jars' so the code can take or replace AA letters on a ‘like for like’ basis
#recall that these related to the ‘in between’ codons are coded into A's/a's (alpha helices), B's/b's(beta sheets) & t's
#the AA letters are allocated (using discretion) to jars using the Berg Tymoczko Stryer table of alpha/beta/turns frequencies
jarA=[E,Q,H,D,R,K]#alpha helix—H'philic Amino Acids (AAs)
jarB=[Y,T,R,H,G,S]#beta sheets—H'philic AAs
jara=[A,C,L,F,W,M]#alpha helix—H'phobic AAs
jarb=[V,I,F,T,L]#beta sheets—H'phobic AAs
jart=[G,S,D,N,P]#turns—no specific hydropathy assumed
#these are the reduced volume AAs to be selected on testing for steric clash, AAs 1-2 are low volume, letter 3 is high volume.
jarAr=[E,D,R]#alpha helix-H'philic Amino Acids (AAs)
jarBr=[T,S,W]#beta sheets-H'philic AAs
jarar=[A,C,F]#alpha helix-H'phobic AAs
jarbr=[V,T,L]#beta sheets-H'phobic AAs
#jart=[G,S,D,N,P]#turns-same so no jartr required
#AAs for orbit letters are also placed in jars' so the code can also take or replace AA letters on a ‘like for like’ basis
#recall that the orbit letters are one of J,Z,X & U ie the orbit letters are J(_A_),Z(_G_),X(_C_),U(_T_) and we
#designate AAs swapped for J,Z,X & U with lower case aa's so the computer does not confuse with the non orbit letters in the program
#orbit codons have a central letter as an orbit base—based on the Natures Code paper scheme
jarOTP=[m]#T=U+
jarOCP=[s,p,t]#C=X+
jarOAP=[y,h,q,n,k,d,e]#A=J+
jarOGP=[r,s,g]#G=Z+
jarOTp=[f,l,i,m,v]#T=u−
jarOCp=[s,p,t,a]#C=x−
jarOAp=[y,h]#A=j−
jarOGp=[c,w,s,g]#G=z−
#this dictionary is used to convert values back to AAs towards end of this code
jarDict={“G”: 60.1,“A”: 88.6,“S”: 89.1,“C”: 108.5,“D”:111.1,“P”:112.7,“N”: 114.1,“T”: 116.1,“E”:138.4,“V”:140.1, “Q”: 143.8,“H”: 153.2,“M”: 162.9,“I”: 166.6,“L”:166.7,“K”:168.6,“R”: 173.4,“F”: 189.9,“Y”:193.6,“W”:227.8}
jarDictO={“g”: 60,“a”: 88,“s”: 89,“c”: 108,“d”:111,“p”:112,“n”: 114,“t”: 116,“e”:138,“v”:140, “q”: 143,“h”: 153,“m”: 162,“i”: 166,“1”: 167,“k”:168,“r”: 173,“f”: 189,“y”:193,“w”:227}
#this code block takes the pentgrid1 list of orbit values mixed with presumed intermediate alpha AAs, beta AAs or turn AAs and converts them to AA
#letters so that PentrGrid1 becomes a homogenous list of AA letters (upper and lower case) which can then be tested for steric clashes
#those letters are upper and lower case as they originate from orbit letters (lower case AAs) and non orbit (intermediates) (upper case letters)
#the reason why they are separated is that this algorithm conducts multiple loops and may take ‘over swap’ AA's unless they are lower/upper case
for i in range(len(pentGrid1)−1):
   if pentGrid1[i]==‘A’:
      pentGrid1[i]=random.choice(jarA)
   elif pentGrid1[i]==‘B’:
      pentGrid1 [i]=random.choice(jarB)
   elif pentGrid1[i]==‘a’:
      pentGrid1[i]=random.choice(jara)
   elif pentGrid1[i]==‘b’:
      pentGrid1[i]=random.choice(jarb)
   elif pentGrid1[i]==‘t’:
      pentGrid1[i]=random.choice(jart)
   elif pentGrid1[i]==‘U’:
      pentGrid1[i]=random.choice(jarOTP)
   elif pentGrid1[i]==‘X’:
      pentGrid1[i]=random.choice(jarOCP)
   elif pentGrid1[i]==‘J’:
      pentGrid1[i]=random.choice(jarOAP)
   elif pentGrid1[i]==‘Z’:
      pentGrid1[i]=random.choice(jarOGP)
   elif pentGrid1[i]==‘u’:
      pentGrid1[i]=random.choice(jarOTp)
   elif pentGrid1[i]==‘x’:
      pentGrid1[i]=random.choice(jarOCp)
   elif pentGrid1[i]==‘j’:
      pentGrid1[i]=random.choice(jarOAp)
   elif pentGrid1[i]==‘z’:
      pentGrid1[i]=random.choice(jarOGp)
   print(pentGrid1)
   pentGrid2=pentGrid1
   continue
#this code block tests each AA letter and compares it with its neighbouring letter for a Steric clash which is performed
#by comparing the volumes between neighbours—if the volume of any two consecutive neighbouring letters is >300 then
#the code replaces the letter ‘for that iteration’ (i) from the same ‘jar’ of AA letters for i in range (len(pentGrid2)−1):
   if pentGrid2[i]==E or pentGrid2[i]==Q or pentGrid2[i]==H or pentGrid2[i]==D\
      or pentGrid2[i]==R or pentGrid2[i]==K\
         and pentGrid2[i]+pentGrid2[i+1]>300 and pentGrid2[i]>135:
         pentGrid2[i]=random.choice(jarAr)
   elif pentGrid2[i]— Y or pentGrid2[i]==T \
      and pentGrid2[i]+pentGrid2[i+1]>300 and pentGrid2[i]>135:
         pentGrid2[i]=random.choice(jarBr)
   elif pentGrid2[i]==A or pentGrid2[i]==C or pentGrid2[i]==L or pentGrid2[i]==F or pentGrid2[i]==W or pentGrid2[i]==M\
   and pentGrid2[i]+pentGrid2[i+1]>300 and pentGrid2[i]>135:
      pentGrid2[i]=random.choice(jarar)
   elif pentGrid2[i]==V or pentGrid2[i]==I or pentGrid2[i]==F or pentGrid2[i]==T or pentGrid2[i]==L\
      and pentGrid2[i]+pentGrid2[i+1]>300 and pentGrid2[i]>135:
         pentGrid2[i]=random.choice(jarbr)
   elif pentGrid2[i]==G or pentGrid2[i]==S or pentGrid2[i]==D or pentGrid2[i]==N or pentGrid2[i]==P\
      and pentGrid2[i]+pentGrid2[i+1]>300 and pentGrid2[i]>135:
         pentGrid2[i]=random.choice(jart)
   #continue
print(pentGrid2)
#this code block converts the volume values back to AA letters for the final print out-note orbit peptides are left as volumes
#because they need to be replaced with nucleotides whose central letter is an orbit letter unlike the intermediate peptides
#which can be replaced from codon jars according to the Amino Acids—DNA Codon table without such constraint
for i in range(len(pentGrid2)−1):
   for key, value in jarDict.items( )
      try: pentGrid2[i]+pentGrid2[i+1] is float
      except:
         continue
      if value in pentGrid2:
         index=pentGrid2.index(value)
      pentGrid2[index]=key
print(pentGrid2)
   Annexure E: Sequence of Amino Acids being Output of Python Program in Annexure D (Output of Stage 3)
   [The final Amino Acid sequence appears in bold—the previous lines of output represent the course of evaluation by the program to process the GGF walk orbit triplets and ‘intermediate’ triplets]
[114, 173.4, 153.2, 153.2, 166.6, 138, 140.1, 166.6, 168.6, 153.2, 114, 143.8, 153.2, 111.1, 153.2, 173, 116.1, 173.4, 173.4, 153.2, 138, 143.8, 111.1, 168.6, 112.7, 153, 60.1, 116.1, 89.1, 193.6, 193, 88.6, 227.8, 166.7, 88.6, 193, 166.7, 60.1, 111.1, 111.1, 193, 153.2, 143.8, 143.8, 116.1, 227,‘b’,‘b’,‘A’,‘A’,‘Z’,‘A’,‘A’,‘t’,‘B’,‘Z’,‘B’,‘B’,‘B’,‘B’,‘J’,‘A’,‘A’,‘A’,‘t’,‘a’,‘J’,‘B’,‘B’, ‘B’,‘B’,‘j’,‘a’,‘a’,‘a’,‘a’]
[114, 111.1, 173.4, 138.4, 140.1, 138, 116.1, 116.1, 138.4, 138.4, 114, 173.4, 173.4, 111.1, 111.1, 173, 116.1, 173.4, 138.4, 173.4, 138, 138.4, 138.4, 168.6, 112.7, 153, 89.1, 166.7, 114.1, 116.1, 193, 108.5, 189.9, 108.5, 189.9, 193, 88.6, 112.7, 173.4, 138.4, 193, 173.4, 138.4, 111.1, 116.1, 227, 166.7, 140.1, 173.4, 173.4, 60, 111.1, 111.1, 114.1, 138.4, 173, 60.1, 89.1, 112.7, 173.4, 111, 111.1, 138.4, 173.4, 111.1, 189.9, 193, 112.7, 89.1, 116.1, 227.8, 153, 108.5, 108.5, 108.5,‘a’]
[114,‘D’,‘R’,‘E’,‘V’, 138,‘T’,‘T’,‘E’,‘E’, 114,‘R’,‘R’,‘D’,‘D’, 173,‘T’,‘R’,‘E’,‘R’, 138, ‘E’,‘E’,‘K’,‘P’, 153,‘S’,‘L’,‘N’,‘T’, 193,‘C’,‘F’,‘C’,‘F’, 193,‘A’,‘P’,‘R’,‘E’, 193,‘R’,‘E’, ‘D’,‘T’, 227,‘L’,‘V’,‘R’,‘R’, 60,‘D’,‘D’,‘N’,‘E’, 173,‘G’,‘S’,‘P’,‘R’, 111,‘D’,‘E’,‘R’,‘D’, ‘F’, 193,‘P’,‘S’,‘T’,‘W’, 153,‘C’,‘C’,‘C’,‘a’]>>>
   Annexure F: Python Program to Convert Amino Acid Sequence being Output from Codeweaver Stage 3 to a DNA Sequence Encoding that Sequence (Representing the Peptide Chain in Respect of the GGF Walk Shadowing the Biocurve) (Stage 4)
(#=comments)
#CODEWEAVER STAGE 4 PYTHON CODE
import random
#This code randomly replaces each amino acid in the peptide chain extrapolated in Codeweaver Stage 3
#for a codon to create a DNA sequence for a biologic drug or vaccine—the peptide chain is the pentGrid4 list below
pentGrid4=[114,‘D’,‘R’,‘E’,‘V’, 138,‘T’,‘T’,‘E’,‘E’, 114,‘R’,‘R’,‘D’,‘D’, 173,‘T’,‘R’,‘E’,‘R’, 138, ‘E’,‘E’,‘K’,‘P’, 153,‘S’,‘L’,‘N’,‘T’, 193,‘C’,‘F’,‘C’,‘F’, 193,‘A’,‘P’,‘R’,‘E’, 193,‘R’,‘E’,‘D’,‘T’, 227,‘L’,‘V’,‘R’,‘R’, 60,‘D’,‘D’,‘N’,‘E’, 173,‘G’,‘S’,‘P’,‘R’, 111,‘D’,‘E’,‘R’,‘D’,‘F’, 193,‘P’,‘S’, ‘T’,‘W’, 153,‘C’,‘C’,‘C’,‘a’]
#this dictionary only lists amino acid volumes for formatting reasons in this code—otherwise they are not needed
jarDict4={“G”: 60.1,“A”: 88.6,“S”: 89.1,“C”: 108.5,“D”:111.1,“P”:112.7,“N”: 114.1,“T”: 116.1,“E”:138.4,“V”:140.1, “Q”: 143.8,“H”: 153.2,“M”: 162.9,“I”: 166.6,“L”:166.7,“K”:168.6,“R”: 173.4,“F”: 189.9,“Y”:193.6,“W”:227.8}
jarDictO4={60:“G”,88:“A”,89:“S”,108:“C”,111:“D”,112:“P”,114:“N”,116:“T”,138:“E”,140:“V”, 143: “Q”,153:“H”,162:“M”,166: “I”,167:“L”,168:“K”,173:“R”,189:“F”,193:“Y”,227:“W”}

jarG = [‘GGT’, ‘GGC’, ‘GGA’, ‘GGG’]
jarA = [‘GCT’, ‘GCC’, ‘GCA’, ‘GCG’]
jarC = [‘TGT’, ‘TGC’]
jarD = [‘GAT’, ‘GAC’]
jarP = [‘CCT’, ‘CCC’, ‘CCA’, ‘CCG’]
jarN = [‘AAT’, ‘AAC’]
jarT = [‘ACT’, ‘ACC’, ‘ACA’, ‘ACG’]
jarE = [‘GAA’, ‘GAG’]
jarV = [‘GTT’, ‘GTC’, ‘GTA’, ‘GTG’]
jarQ = [‘CAA’, ‘CAG’]
jarH = [‘CAT’, ‘CAC ]
jarM = [‘ATG’]
jarI = [‘ATT’, ‘ATC’, ‘ATA’]
jarL = [‘TTA’, ‘TTG’, ‘CTT’, ‘CTC’, ‘CTA’,
‘CTG’]
jarK = [‘AAA’, ‘AAG’]
jarR = [‘AGA’, ‘AGG’]
jarF = [‘TTT’, ‘TTC’]
jarY = [‘TAT’, ‘TAC’]
jarW = [‘TGG’]
jar60 = [‘GGT’, ‘GGC’, ‘GGA’, ‘GGG’]
jar88 = [‘GCT’, ‘GCC’, ‘GCA’, ‘GCG’]
jar89 = [‘TCT’, ‘TCC’, ‘TCA’, ‘TCG’]
jar108 = [‘TGT’, ‘TGC’]
jar111 = [‘GAT’, ‘GAC’]
jar112 = [‘CCT’, ‘CCC’, ‘CCA’, ‘CCG’]
jar114 = [‘AAT’, ‘AAC’]
jar116 = [‘ACT’, ‘ACC’, ‘ACA’, ‘ACG’]
jar138 = [‘GAA’, ‘GAG’]
jar140 = [‘GTT’, ‘GTC’, ‘GTA’, ‘GTG’]
jar143 = [‘CAA’, ‘CAG’]
jar153 = [‘CAT’, ‘CAC’]
jar162 = [‘ATG’]
jar166 = [‘ATT’, ‘ATC’, ‘ATA’]
jar167 = [‘TTA’, ‘TTG’, ‘CTT’, ‘CTC’, ‘CTA’,
‘CTG’]
jar168 = [‘AAA’, ‘AAG’]
jar173 = [‘AGA’, ‘AGG’]
jar189 = [‘TTT’, ‘TTC’]
jar193 = [‘TAT’, ‘TAC’]
jar227 = [‘TGG’]

#this code converts the amino acid sequence—pentGrid4—encoded by an unknown theoretical DNA sequence to an actual sequence
#by randomly choosing a codon from one of the 20 respective amino acid jar groups above
for i in range (len(pentGrid4)−1):
   if pentGrid4[i]==‘G’:
      pentGrid4[i]=random.choice(jarG)
   elif pentGrid4[i]==‘A’:
      pentGrid4[i]=random.choice(jarA)
   elif pentGrid4[i]==‘S’:
      pentGrid4[i]=random.choice(jarS)
   elif pentGrid4[i]==‘C’:
      pentGrid4[i]=random.choice(jarC)
   elif pentGrid4[i]==‘D’:
      pentGrid4[i]=random.choice(jarD)
   elif pentGrid4[i]==‘P’:
      pentGrid4[i]=random.choice(jarP)
   elif pentGrid4[i]==‘N’:
      pentGrid4[i]=random.choice(jarN)
   elif pentGrid4[i]==‘T’:
      pentGrid4[i]=random.choice(jarT)
   elif pentGrid4[i]==‘E’:
pentGrid4[i]=random.choice(jarE)
   elif pentGrid4[i]==‘V’:
pentGrid4[i]=random.choice(jarV)
   elif pentGrid4[i]==‘Q’:
      pentGrid4[i]=random.choice(jarQ)
   elif pentGrid4[i]==‘H’:
pentGrid4[i]=random.choice(jarH)
   elif pentGrid4[i]==‘M’:
      pentGrid4[i]=random.choice(jarM)
   elif pentGrid4[i]=‘I’:
pentGrid4[i]=random.choice(jarI)
   elif pentGrid4[i]==‘L’:
      pentGrid4[i]=random.choice(jarL)
   elif pentGrid4[i]==‘K’:
pentGrid4[i]=random.choice(jarK)
   elif pentGrid4[i]==‘R’:
pentGrid4[i]=random.choice(jarR)
   elif pentGrid4[i]==‘F’:
      pentGrid4[i]=random.choice(jarF)
   elif pentGrid4[i]==‘Y’:
      pentGrid4[i]=random.choice(jarY)
   elif pentGrid4[i]==‘W’:
      pentGrid4[i]=random.choice(jarW)
   elif pentGrid4[i]==60:
      pentGrid4[i]=random.choice(jar60)
   elif pentGrid4[i]==88:
      pentGrid4[i]=random.choice(jar88)
   elif pentGrid4[i]==89:
      pentGrid4[i]=random.choice(jar89)
   elif pentGrid4[i]==108:
      pentGrid4[i]=random.choice(jar108)
   elif pentGrid4[i]==111:
      pentGrid4[i]=random.choice(jar111)
   elif pentGrid4[i]==112:
      pentGrid4[i]=random.choice(jar112)
   elif pentGrid4[i]==114:
pentGrid4[i]=random.choice(jar114)
   elif pentGrid4[i]==116:
      pentGrid4[i]=random.choice(jar116)
   elif pentGrid4[i]==138:
      pentGrid4[i]=random.choice(jar138)
   elif pentGrid4[i]==140:
      pentGrid4[i]=random.choice(jar140)
   elif pentGrid4[i]==143:
      pentGrid4[i]=random.choice(jar143)
   elif pentGrid4[i]==153:
      pentGrid4[i]=random.choice(jar153)
   elif pentGrid4[i]==162:
      pentGrid4[i]=random.choice(jar162)
   elif pentGrid4[i]==166:
      pentGrid4[i]=random.choice(jar166)
   elif pentGrid4[i]==167:
      pentGrid4[i]=random.choice(jar167)
   elif pentGrid4[i]==168:
      pentGrid4[i]=random.choice(jar168)
   elif pentGrid4[i]==173:
      pentGrid4[i]=random.choice(jarR)
   elif pentGrid4[i]==189: pentGrid4[i]=random.choice(jar189)
   elif pentGrid4[i]==193:
      pentGrid4[i]=random.choice(jar193)
   elif pentGrid4[i]==227:
      pentGrid4[i]=random.choice(jar227)
#this code repeats the above process to replace the remaining amino acids not converted
#(nb. takes 30 seconds to process—# for higher throughput the code below would be
#replaced for computer speed purposes
   for key, value in jarDict4.items( )
      if value in pentGrid4:
         continue
      print(pentGrid4)
         Annexure G: Sequence of DNA being Output of Python Program in Annexure F

[The final DNA sequence appears in bold—the previous sequence is the output of Codeweaver Stage 3 representing the Amino Acid or peptide chain which is extrapolated to produce the GGF walk shadowing the Biocurve] (Output of Stage 4)
Amino acid sequence:
[114,‘D’,‘R’,‘E’,‘V’, 138,‘T’,‘T’,‘E’,‘E’, 114,‘R’,‘R’,‘D’,‘D’, 173,‘T’,‘R’,‘E’,‘R’, 138,‘E’,‘E’,‘K’, ‘P’, 153,‘S’,‘L’,‘N’,‘T’, 193,‘C’,‘F’,‘C’,‘F’, 193,‘A’,‘P’,‘R’,‘E’, 193,‘R’,‘E’,‘D’,‘T’, 227,‘L’,‘V’, ‘R’,‘R’, 60,‘D’,‘D’,‘N’,‘E’, 173,‘G’,‘S’,‘P’,‘R’, 111,‘D’,‘E’,‘R’,‘D’,‘F’, 193,‘P’,‘S’,‘T’,‘W’, 153, ‘C’,‘C’,‘C’,‘a’]
[numbers represent peptides generated from orbit nucleotides and those peptides are ascertained by reference to the dictionary used in stage 3 and 4—this is the dictionary used in Stage 4:
jarDict04={60:“G”,88:“A”,89:“S”,108:“C”,111:“D”,112:“P”,114:“N”,116:“T”,138:“E”,140:“V”, 143: “Q”,153:“H”,162:“M”,166:“I”,167:“L”,168:“K”,173:“R”,189:“F”,193:“Y”,227:“W”}
So the above Amino acid sequence is converted into the DNA sequence below by the Codeweaver Stage 4 Python Code in Annexure F

DNA sequence
[‘AAC’, ‘GAT’, ‘AGG’, ‘GAA’, ‘GTG’, ‘GAA’, ‘ACT’,
‘ACA’, ‘GAG’, ‘GAA’, ‘AAT’, ‘AGA’, ‘AGG’, ‘GAT’,
‘GAC’, ‘AGA’, ‘ACG’, ‘AGA’, ‘GAA’, ‘AGA’, ‘GAG’,
‘GAA’, ‘GAG’, ‘AAA’, ‘CCG’, ‘CAC’, ‘TCA’, ‘TTA’,
‘AAT’, ‘ACC’, ‘TAC’, ‘TGC’, ‘TTC’, ‘TGC’, ‘TTC’,
‘TAT’, ‘GCC’, ‘CCC’, ‘AGG’, ‘GAG’, ‘TAT’, ‘AGA’,
‘GAG’, ‘GAT’, ‘ACA’, ‘TGG’, ‘TTA’, ‘GTT’, ‘AGA’,
‘AGA’, ‘GGC’, ‘GAT’, ‘GAC’, ‘AAT’, ‘GAG’, ‘AGG’,
‘GGA’, ‘TCT’, ‘CCA’, ‘AGG’, ‘GAC’, ‘GAC’, ‘GAA’,
‘AGG’, ‘GAC’, ‘TTT’, ‘TAT’, ‘CCC’, ‘TCT’, ‘ACG’,
‘TGG’, ‘CAC’, ‘TGT’, ‘TGC’, ‘TGT’, ‘a’]

### Examples

As shown in FIG. 4, the GGF algorithm was applied to the Human Mitochondrion and compared to a random A, G, C & T GGF print (left). The middle diagram allows the GGF print for Drosophila melanogaster fruit fly to be compared to the framework of wing or leg.

The GGF method can be used in the following ways as described:

(1) an Analytical Probe, Test or Sensor Tool

(1) A probe, test or sensor to analyse DNA, RNA, proteins or polypeptides or other macromolecular sequences (“Code Sequences”) to recognize, detect, analyse, store or otherwise process images, configurations, patterns, graphic signatures or representations of code execution or encoding or other code mapping or reverse mapping which may be inherent in the order of Code Sequences in order to interpret and provide systematic representations of the particular macromolecular code analysed. Such representations have potential to be significant to the biology of the organism or entity including to provide representations indicating morphology, cell differentiation, protein synthesis, transcription, translation, genetics, reproduction, protein folding or other behaviour, cell metabolism such as binary sequences of genetic, protein or other circuits which deploy morphology incidental to cell metabolism or possibly indicative of cell metabolism itself including where such order of macromolecules represents a code or nascent code or fragments of a code, pseudo code or obsolete code as the case may be. These representations would in turn be of use in bioinformatics, biological, biomedical, medical, veterinary, biochemical, biotechnological, pharmaceutical fields, vaccine development, genetic testing and allied sciences and applied sciences (or related fields) whether for research, development, clinical, commercial, industrial or other uses.

Methodology for such probes, tests or sensors could include:

(a) Such probes, tests or sensors could follow protocols where GGF Motif databases are created during the procedure and interrogated or interpreted to provide guidance for future biological projects.

(b) Such probes, tests or sensors could rely on generic GGF Motif databases to classify DNA/RNA or other sequences—for example, a number of generic GGF motifs across different motifs such as helices, sheets or specific patterns have been noted which already provide an immediate simple classification method—note it is postulated that the typical alpha helices and beta sheets in proteins match the helices or sheet GGF motifs being detected in GGF motif print outs (See FIG. 3) Thus, a comprehensive GGF motif library and database with interpreted one to one or one to many matching biological features could be used as a predictive tool in research, development, clinical practice and commerce to provide analytical or detection methods for determining likely gene expression, transcription, translation, protein synthesis, cell differentiation or morphology or even metabolic function given that GGF motifs seem to bear relation to actual molecular structures that would be expected to be expressed from genome sequences run by the GGF algorithm;

(c) As an analytical tool for formulating new models, schemes, genetic codes or epigenetic codes by producing code systems or schemes that provide systematic decoding schemes for gene transcription, translation, protein synthesis, housekeeping genes, histone codes, hox codes or other genetic/epigenetic codes;

(d) Such probes, tests or sensors could be used as a library or database populating and/or formatting tool to:
(i) increase quality of libraries or databases (especially where fidelity of samples is questionable) eg to detect repeater sequences, palindromic sequences, variability criteria or where statistical noise can mask features sought or where the detection method may sustain statistical noise eg noise associated with mass spectrometer DNA analysis for high throughput DNA sequencing;
(ii) reduce high throughput computer processing/running time (measured by polynomial time, exponential time or other running times) where DNA analysis is dependent on numerical/iterative analysis. For example, A GGF motif library could be assembled to act as a probe, test or sensor to detect randomness or non randomness in DNA—or heterogeneity or homogeneity. There are examples of statistical models being constructed to detect amino acid variability in antibodies (such plots have been called Wu-Kabat Plots by Professor Ted Steele and his colleagues5 which disclosed Wu Kabat Structures indicating highly non random patterns). Another example could be introducing GG Motif Regression Analysis as an improved method over linear regression analysis.

(e) An alternative method to the standard DNA hybridization tool-standard DNA hybridization is a “method of determining the similarity of DNA from different sources”, e.g different bacterial species are put together and the extent to which double hybrid stands are formed is estimated. The greater the tendency to form double hybrid molecules, the greater the extent of complementary base sequences i.e. gene similarity. The method is one way of determining genetic relationships of species. The same principle applies when using DNA probes to search a particular base sequence in a sample of DNA e.g. when screening a DNA library for a particular cloned fragment or in a DNA microarray technology. The technique of allele-specific oligonucleotide hybridization is used to test DNA from individuals to determine whether they are carriers of disease—causing alleles. Oxford Dictionary of Biology. Thus, a GGF motif database could augment or replace these DNA hybridization laboratory testing procedures and new protocols and procedures advantageous to existing standard hybridization protocols and procedures.

(f) the operation of Artificial Intelligence systems which use GGF motif libraries, databases or elements, or GGF based mathematical models to generate candidate analogues of gene sequences to be generated for use in genetic engineering, genome editing or other synthetic genetic or epigenetic sequencing;

(g) the operation of modelling systems which use GGF motif libraries or databases or elements, or GGF based mathematical models to generate candidate analogues of gene sequences to be generated for use in genetic engineering, genome editing or other synthetic genetic or epigenetic sequencing;

(h) Tests, probes, sensors or other detection processes or devices which utilize the GGF Motif analysis or GGF Motif Libraries or Databases or other sequencing programs with GGF Motif elements which could be housed in fixed or mobile lab units or miniaturized into a test kit or sensor given sufficient memory in the integrated circuit used in any such sensor or test kit—such a test kit might be a paper based test kits using a range of RNA engineered set of ‘dry’ RNA molecules to test base sequences in the field. Such a fixed or mobile unit or test kit might be used in relation to other GGF applications listed in this application;

As shown in FIG. 5 the DNA sequences of 2 proteins were both subjected to a GGF print. On the left a GGF print of Glycophorin (has alpha helices) and on the right a GGF print of Porins (typically beta sheets). Thus, the GGF produced a motif of helices for Glycophorin and a ‘sheet of dots’ motif for Porins. What is striking is that on reversing direction of GGF code the Porins gene printed a helical cord with some similarity to the Glycophorin gene's normal helical print whilst Glycophorin's reverse print was a ‘sheet of dots’ patterned not unlike the dots in the Porin's beta sheets motif. Equally striking was a GGF frame test performed on both Bacteriophage G4 virus and Covid-19 virus both of which displayed similar generic GGF motifs namely ‘sheet of dots’ motifs on one base GGF frame shifts possibly indicating similar generic transcription schemes in each viral genome encoding key generic elements of viral proteins, similar to Porins Beta Sheets—See Panel 3 FIG. 5.

(2) As Datasets Databases or Libraries

Different motifs for different DNA sequences can be compiled into a library or database of GGF motifs which could be compiled into legends or tables with GGF Motif elements matching DNA sequences, genes, morphologies, exons, introns, mutations, neoplasms, metaplasias, irregular proteins, isoform proteins, dysfunctional proteins, or other genetic or biological features or abnormalities or defects. Any such legend or table could form a type of ‘Rosetta stone’ allowing valuable interpretation of DNA, RNA, polypeptide or other sequences, gene transcription fate maps, translation, gene regulation maps, gene circuits, protein circuits, metabolic maps, or other biological circuits, protein synthesis and folding analysis, cell signalling, cell differentiation, morphologies or a general scheme for different gene expression propensities for different types of gene expression for research, development, clinical or commercial use.

The uses of such datasets, databases or libraries could include:

(i) Compiling a new genetic defect risk profile database or library to gauge the potential cancer, disease or other risk factors that GGF motifs printed from genomes provide based on correlating genome sequence, GGF motif (or generic motif) with clinical links to cancer, disease or other dysfunction or feature or statistical correlation indicated by such motifs (the difference between existing genetic defect databases and this new database is that this database can be predictive or referential because it provides a criterion of analysis based on the image signature generated from the pattern of DNA sequence resulting from a GGF print and comparison to a set of known generic GGF motifs which have been considered as indicative of cancer, disease or other dysfunction—as opposed to a simple match of sequences by statistical matching or correlation techniques such as linear regression analysis.) For example, the GGF formatting and algorithm could be used in GGF Motif Regression analysis (the GGF Motif is regressed to a generic or target GGF motif rather than a line) and would aim to be predictively prospective prior to clinical evidence rather than retrospective based on past clinical evidence.

(ii) Compiling a new genetic parts database or RNA interaction library (e.g. RNA features, folding kinetics etc) to predict sequences expressing potential parts or to gauge the potential for the target genetic or other sequences to produce targeted genetic parts for use in biological or biomedical modelling, cellular programming, design of genetic, cell or protein circuits, genetic engineering, genome editing, drug or supplemental design to treat cancer, disease, vitamin or mineral deficiencies or other health supplements or other genetic or biological design that GGF motifs printed from genomes provide based on correlating genome sequence, GGF motif (or generic motif) to the genetic part or parts indicated by such motifs—examples of genetic parts are RNA scaffolds, riboswitches, RNAi, sRNA, sRNA circuit vectors, trans splicing ribozymes and self splicing introns;

(iii) Compiling new genomics, bioinformatics or other databases or libraries to classify target genetic or other sequences to produce information, models and other research/development tools relevant to biochemistry, molecular biology, organic chemistry, genealogy, oncology, biomedical sciences, medical sciences, genetic or other biological sequence, evolutionary theory or history or origin of life theory or history that GGF motifs printed from genomes provide based on correlating genome sequence, GGF motif (or generic motif) to the biological part, function, statistical correlation or other biological feature indicated by such motifs;

(iv) improving accuracy of prediction with respect to genome engineering or editing outcomes including high throughput screening of genes facilitating, improving and hastening the processes and protocol associated with genome engineering;

(3) As a Design Tool.

The use of GGF motif datasets, libraries, databases or elements or statistical methods or libraries, databases or elements which utilize GGF Motifs to design, generate, calibrate, fine tune or otherwise supply DNA/RNA, amino acid or other sequences to fulfil metabolic or morphological mechanisms, cell differentiation or cell signalling functions, genetic products, genetic, protein or other circuits (designed or otherwise), produce therapeutic products, treatments, biomedical science tools, medical science tools, bioinformatic tools, computational tools, models, medicine or chemicals, facilitate genetic engineering, genome editing, tests, sensors or other biological products or procedures by various methods using GGF motifs including:
(a) use of cellular models for RNA control over protein regulation;
(b) targeting suitable gene sequences to program cellular function;
(c) designing a repertoire or repertoires of genetic parts (natural or synthetic);
(d) creating RNA circuits to test candidate DNA/RNA sequences where matching of GGF motifs with candidate sequences can achieve better results or hasten modelling and design processes;
(e) using linear regression analysis, transcriptional regulation analysis, translation analysis, expression analysis or a new proposed method GGF motif regression analysis (instead of a straight line targeted by numerical iterations to target correlated sub data set—curve fitting the GGF motif can become the target ‘curve’ to be fitted and such technique could be termed “Regression to the Motif”)

Although the description above contains precise specifications these are not to be interpreted as limiting the scope of the invention but as merely providing illustrations and demonstrations of some of the presently preferred embodiments of this invention. For example, two prime embodiments involved forms of the GGF Method involving rotational symmetry where 2π is divided into radial intervals such that rotational symmetry and reflective symmetry is involved. However, it may be that the user may wish to use configurations that are not symmetrical—for example, to eliminate likely genetic outcomes or other uses.

Thus, the scope of the invention should be determined by the appended claims and their legal equivalents rather than by the embodiments and examples given.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

Attachments of a Preferred Embodiment being a Matematica Program or Code Containing the GGF Algorithm

```
Attachment A- MATHEMATICA CODE PRINTOUT FOR GGF ALGORITHM n=5 and
{r1,r2,r3,r4} - {2,4,6,8} INCLUDING GGF MOTIF OUTPUT SHOWN AS FIG. 6
SetDirectory[ ]
phagegenome = Import[″C: /Users/chagan.AUMINCO/My Documents/_A_A_A_A_M/phagedata.txt″]}
phageletters = Characters[phagegenome];
FormattedBases = Partition[phageletters, 14];
FourteenthBases = Drop[FormattedBases, None, 13];
FlatFourteen = Flatten[FourteenthBases];
BaseTenParts = Partition[FlatFourteen, 10];
FourteenBlocksForty = Flatten[Table[BaseTenParts, 14]];
FormattedBases = Gather[Partition[FourteenBlocksForty, 10]];
Flatten[FormattedBases];
ToExpression[%];
$Angles = ReplaceAll [ \{ a \to \frac{2 Pi}{5} , c \to \frac{4 Pi}{5} , g \to \frac{6 Pi}{5} , t \to \frac{8 Pi}{5} \} ] [ \% ]$
Map[AngleVector, Angles];
DownPhage = Accumulate[%]
(*ListPlot[DownPhage]*)
Protect[DownPhage]
ClearAll[phageletters, FormattedBases]
phagegenome = Import[″C:/Users/chagan.AUMINCO/My Documents/_A_A_A_A_M/phagedata.txt″];
phageletters = Characters[phagegenome];
phageletters = Characters[phagegenome];
FormattedBases = Partition[phageletters, 14];
FourteenthBases = Drop[FormattedBases, None, 13];
FlatFourteen = Flatten[FourteenthBases];
BaseTenParts = Partition[FlatFourteen, 10];
FourteenBlocksForty = Flatten[Table[BaseTenParts, 14]];
FormattedBases = Gather[Partition[FourteenBlocksForty, 10]];
Flatten[FormattedBases];
ToExpression[%];
$AnglesM = ReplaceAll [ \{ t \to \frac{2 Pi}{5} , g \to \frac{4 Pi}{5} , c \to \frac{6 Pi}{5} , a \to \frac{8 Pi}{5} \} ] [ \% ]$
Map[AngleVector, AnglesM];
UpPhage = Accumulate[%]
ListPlot[UpPhage]
ListPlot[DownPhage]
C:\Users\chagan.AUMINCO
$\begin{matrix}\{ \frac{4 π}{5} , \frac{8 π}{5} , \frac{8 π}{5} , \frac{8 π}{5} , \frac{8 π}{5} , \frac{8 π}{5} , \frac{4 π}{5} , \frac{2 π}{5} , \frac{2 π}{5} , \frac{4 π}{5} , \frac{4 π}{5} , \frac{8 π}{5} , \frac{8 π}{5} , \frac{8 π}{5} , \frac{8 π}{5} , \frac{8 π}{5} , \frac{4 π}{5} , \frac{2 π}{5} , \frac{2 π}{5} , \frac{4 π}{5} , \frac{4 π}{5} , \frac{8 π}{5} , \frac{8 π}{5} , \frac{8 π}{5} , \frac{8 π}{5} , \frac{8 π}{5} , \frac{4 π}{5} , \frac{2 π}{5} , \\ \frac{2 π}{5} , \frac{4 π}{5} , \frac{4 π}{5} , \frac{8 π}{5} , \frac{8 π}{5} , \frac{8 π}{5} , \frac{8 π}{5} , \frac{8 π}{5} , \frac{4 π}{5} , \frac{2 π}{5} , \frac{2 π}{5} , \frac{4 π}{5} , \frac{4 π}{5} , \frac{8 π}{5} , \frac{8 π}{5} , \frac{8 π}{5} , \frac{8 π}{5} , \frac{8 π}{5} , \frac{4 π}{5} , \frac{2 π}{5} , \frac{2 π}{5} , \frac{4 π}{5} , \frac{4 π}{5} , \frac{8 π}{5} , \frac{8 π}{5} , \frac{8 π}{5} , \frac{8 π}{5} , \\ \frac{8 π}{5} , \frac{4 π}{5} , \frac{2 π}{5} , \frac{2 π}{5} , \frac{4 π}{5} , \frac{4 π}{5} , \frac{8 π}{5} , \frac{8 π}{5} , \frac{8 π}{5} , \frac{8 π}{5} , \frac{8 π}{5} , \frac{4 π}{5} , \frac{2 π}{5} , \frac{2 π}{5} , \frac{4 π}{5} , \frac{4 π}{5} , \frac{8 π}{5} , \frac{8 π}{5} , \frac{8 π}{5} , \frac{8 π}{5} , \frac{8 π}{5} , \frac{4 π}{5} , \frac{2 π}{5} , \frac{2 π}{5} , , \\ \frac{6 π}{5} , \frac{8 π}{5} , \frac{4 π}{5} , \frac{8 π}{5} , \frac{8 π}{5} , \frac{8 π}{5} , \frac{8 π}{5} , \frac{2 π}{5} , \frac{6 π}{5} , \frac{2 π}{5} , \frac{6 π}{5} , \frac{8 π}{5} , \frac{4 π}{5} , \frac{8 π}{5} , \frac{8 π}{5} , \frac{8 π}{5} , \frac{8 π}{5} , \frac{2 π}{5} , \frac{6 π}{5} , \frac{2 π}{5} , \frac{6 π}{5} , \frac{8 π}{5} , \frac{4 π}{5} , \frac{8 π}{5} , \frac{8 π}{5} , \frac{8 π}{5} , \frac{8 π}{5} , \\ \frac{2 π}{5} , \frac{6 π}{5} , \frac{2 π}{5} , \frac{6 π}{5} , \frac{8 π}{5} , \frac{4 π}{5} , \frac{8 π}{5} , \frac{8 π}{5} , \frac{8 π}{5} , \frac{8 π}{5} , \frac{2 π}{5} , \frac{6 π}{5} , \frac{2 π}{5} , \frac{6 π}{5} , \frac{8 π}{5} , \frac{4 π}{5} , \frac{8 π}{5} , \frac{8 π}{5} , \frac{8 π}{5} , \frac{8 π}{5} , \frac{2 π}{5} , \frac{6 π}{5} , \frac{2 π}{5} , \frac{6 π}{5} , \frac{8 π}{5} , \frac{4 π}{5} , \\ \frac{8 π}{5} , \frac{8 π}{5} , \frac{8 π}{5} , \frac{8 π}{5} , \frac{2 π}{5} , \frac{6 π}{5} , \frac{2 π}{5} , \frac{6 π}{5} , \frac{8 π}{5} , \frac{4 π}{5} , \frac{8 π}{5} , \frac{8 π}{5} , \frac{8 π}{5} , \frac{8 π}{5} , \frac{2 π}{5} , \frac{6 π}{5} , \frac{2 π}{5} , \frac{6 π}{5} , \frac{8 π}{5} , \frac{4 π}{5} , \frac{8 π}{5} , \frac{8 π}{5} , \frac{8 π}{5} , \frac{8 π}{5} , \frac{2 π}{5} , \frac{6 π}{5} \}\end{matrix}$
    large output  show less  show more  show all  set size limit . . .
$\begin{matrix}\{ \{ \frac{1}{4} ( - 1 - \sqrt{5} ) , \sqrt{\frac{3}{8} - \frac{\sqrt{5}}{4}} \} , ( \frac{2}{4} ( - 1 - \sqrt{5} ) + \frac{1}{4} ( - 1 - \sqrt{5} ) , \sqrt{\frac{5}{8} - \frac{\sqrt{5}}{6}} - \sqrt{\frac{5}{8} + \frac{\sqrt{5}}{8}} \} , \{ \frac{1}{4} ( - 1 - \sqrt{5} ) + \frac{1}{2} ( - 1 + \sqrt{5} ) , \sqrt{\frac{5}{8} - \frac{\sqrt{5}}{8}} - \\ 2 \sqrt{\frac{5}{8} + \frac{\sqrt{5}}{8}} \} , \\ \{ \frac{1}{4} ( - 1 - \sqrt{5} ) + \frac{1}{4} ( - 1 + \sqrt{5} ) , \sqrt{\frac{5}{8} - \frac{\sqrt{5}}{8}} - 3 \sqrt{\frac{5}{8} + \frac{\sqrt{5}}{6}} \} , \{ - 1 + \sqrt{5} + \frac{1}{4} ( - 1 - \sqrt{5} ) , \sqrt{\frac{1}{8} - \frac{\sqrt{5}}{8}} - 4 \sqrt{\frac{5}{8} + \frac{\sqrt{5}}{6}} \} , \\ \{ - 1 - \sqrt{5} + \frac{1}{4} ( - 1 - \sqrt{5} ) + \frac{1}{4} ( - 1 - \sqrt{5} ) , \sqrt{\frac{3}{8} - \frac{\sqrt{5}}{8}} - 5 \sqrt{\frac{5}{8} + \frac{\sqrt{5}}{9}} \} , \{ - 1 + \sqrt{5} + \frac{1}{2} ( - 1 - \sqrt{5} ) + \frac{1}{4} ( - 1 - \sqrt{5} ) , 2 \sqrt{\frac{3}{8} - \frac{\sqrt{5}}{8}} - \\ 5 \sqrt{\frac{5}{8} + \frac{\sqrt{5}}{9}} \} , \\ \{ - 1376 + 34 \sqrt{5} + \frac{3}{4} ( - 1 - \sqrt{5} ) + \frac{3}{4} ( - 1 + \sqrt{5} ) + 84 Cos [ Null ] , 337 \sqrt{\frac{3}{8} - \frac{\sqrt{5}}{6}} - 81 \sqrt{\frac{5}{6} + \frac{\sqrt{5}}{8}} + 84 Sin [ Null ] \} , \\ \{ - 1377 + 35 \sqrt{5} + \frac{3}{4} ( - 1 - \sqrt{5} ) + 84 Cos [ Null ] , 337 \sqrt{\frac{5}{8} - \frac{\sqrt{5}}{8}} - 82 \sqrt{\frac{5}{8} + \frac{\sqrt{5}}{8}} + 84 Sin [ Null ] ) \} , \\ \{ - 1377 + 35 \sqrt{5} + \frac{3}{4} ( - 1 - \sqrt{5} ) + \frac{1}{4} ( - 1 + \sqrt{5} ) - 84 Cos [ Null ] , 337 \sqrt{\frac{5}{8} - \frac{\sqrt{5}}{8}} - 83 \sqrt{\frac{5}{8} + \frac{\sqrt{5}}{8}} + 84 Sin [ Null ] \} , \\ \{ - 1377 + 35 \sqrt{5} + \frac{3}{4} ( - 1 - \sqrt{5} ) + \frac{2}{2} ( - 1 + \sqrt{5} ) - 84 Cos [ Null ] , 337 \sqrt{\frac{5}{8} - \frac{\sqrt{5}}{8}} - 84 \sqrt{\frac{5}{8} + \frac{\sqrt{5}}{8}} + 84 Sin [ Null ] \} , \\ \{ - 1377 + 35 \sqrt{5} + \frac{3}{4} ( - 1 - \sqrt{5} ) + \frac{3}{4} ( - 1 + \sqrt{5} ) - 84 Cos [ Null ] , 337 \sqrt{\frac{5}{8} - \frac{\sqrt{5}}{8}} - 85 \sqrt{\frac{5}{8} + \frac{\sqrt{5}}{8}} + 84 Sin [ Null ] \} , \\ \{ - 1378 + 36 \sqrt{5} + \frac{3}{4} ( - 1 - \sqrt{5} ) + 84 Cos [ Null ] , 337 \sqrt{\frac{5}{8} - \frac{\sqrt{5}}{8}} - 84 \sqrt{\frac{5}{8} + \frac{\sqrt{5}}{8}} + 84 Sin [ Null ] \} , \\ \{ - 1379 + 35 \sqrt{5} + 84 Cos [ Null ] , 336 \sqrt{\frac{5}{8} - \frac{\sqrt{5}}{8}} - 84 \sqrt{\frac{5}{8} + \frac{\sqrt{5}}{8}} + 84 Sin [ Null ] \} \}\end{matrix}$
    large output  show less  show more  show all  set size limit . . .
$\begin{matrix}\{ \frac{6 π}{5} , \frac{2 π}{5} , \frac{2 π}{5} , \frac{2 π}{5} , \frac{2 π}{5} , \frac{2 π}{5} , \frac{6 π}{5} , \frac{8 π}{5} , \frac{8 π}{5} , \frac{6 π}{5} , \frac{6 π}{5} , \frac{2 π}{5} , \frac{2 π}{5} , \frac{2 π}{5} , \frac{2 π}{5} , \frac{2 π}{5} , \frac{6 π}{5} , \frac{8 π}{5} , \frac{8 π}{5} , \frac{6 π}{5} , \frac{6 π}{5} , \frac{2 π}{5} , \frac{2 π}{5} , \frac{2 π}{5} , \frac{2 π}{5} , \frac{2 π}{5} , \frac{6 π}{5} , \frac{8 π}{5} , \\ \frac{8 π}{5} , \frac{6 π}{5} , \frac{6 π}{5} , \frac{2 π}{5} , \frac{2 π}{5} , \frac{2 π}{5} , \frac{2 π}{5} , \frac{2 π}{5} , \frac{6 π}{5} , \frac{8 π}{5} , \frac{8 π}{5} , \frac{6 π}{5} , \frac{6 π}{5} , \frac{2 π}{5} , \frac{2 π}{5} , \frac{2 π}{5} , \frac{2 π}{5} , \frac{2 π}{5} , \frac{6 π}{5} , \frac{8 π}{5} , \frac{8 π}{5} , \frac{6 π}{5} , \frac{6 π}{5} , \frac{2 π}{5} , \frac{2 π}{5} , \frac{2 π}{5} , \frac{2 π}{5} , \\ \frac{2 π}{5} , \frac{6 π}{5} , \frac{8 π}{5} , \frac{6 π}{5} , \frac{6 π}{5} , \frac{6 π}{5} , \frac{2 π}{5} , \frac{2 π}{5} , \frac{2 π}{5} , \frac{2 π}{5} , \frac{2 π}{5} , \frac{6 π}{5} , \frac{6 π}{5} , \frac{8 π}{5} , \frac{6 π}{5} , \frac{6 π}{5} , \frac{2 π}{5} , \frac{2 π}{5} , \frac{2 π}{5} , \frac{2 π}{5} , \frac{2 π}{5} , \frac{6 π}{5} , \frac{8 π}{5} , \frac{8 π}{5} , , \\ \frac{4 π}{5} , \frac{2 π}{5} , \frac{6 π}{5} , \frac{2 π}{5} , \frac{2 π}{5} , \frac{2 π}{5} , \frac{2 π}{5} , \frac{8 π}{5} , \frac{4 π}{5} , \frac{8 π}{5} , \frac{4 π}{5} , \frac{2 π}{5} , \frac{6 π}{5} , \frac{2 π}{5} , \frac{2 π}{5} , \frac{2 π}{5} , \frac{2 π}{5} , \frac{8 π}{5} , \frac{4 π}{5} , \frac{8 π}{5} , \frac{4 π}{5} , \frac{2 π}{5} , \frac{6 π}{5} , \frac{2 π}{5} , \frac{2 π}{5} , \frac{2 π}{5} , \frac{2 π}{5} , \\ \frac{8 π}{5} , \frac{4 π}{5} , \frac{8 π}{5} , \frac{4 π}{5} , \frac{2 π}{5} , \frac{6 π}{5} , \frac{2 π}{5} , \frac{2 π}{5} , \frac{2 π}{5} , \frac{2 π}{5} , \frac{8 π}{5} , \frac{4 π}{5} , \frac{6 π}{5} , \frac{4 π}{5} , \frac{2 π}{5} , \frac{6 π}{5} , \frac{2 π}{5} , \frac{2 π}{5} , \frac{2 π}{5} , \frac{2 π}{5} , \frac{8 π}{5} , \frac{4 π}{5} , \frac{8 π}{5} , \frac{4 π}{5} , \frac{2 π}{5} , \frac{6 π}{5} , \\ \frac{2 π}{5} , \frac{2 π}{5} , \frac{2 π}{5} , \frac{2 π}{5} , \frac{8 π}{5} , \frac{4 π}{5} , \frac{6 π}{5} , \frac{4 π}{5} , \frac{2 π}{5} , \frac{6 π}{5} , \frac{2 π}{5} , \frac{2 π}{5} , \frac{2 π}{5} , \frac{2 π}{5} , \frac{8 π}{5} , \frac{4 π}{5} , \frac{8 π}{5} , \frac{4 π}{5} , \frac{2 π}{5} , \frac{6 π}{5} , \frac{2 π}{5} , \frac{2 π}{5} , \frac{2 π}{5} , \frac{2 π}{5} , \frac{2 π}{5} , \frac{4 π}{5} \}\end{matrix}$
    large output  show less  show more  show all  set size limit . . .
$\begin{matrix}\{ \{ \frac{1}{4} \times ( - 1 - \sqrt{5} ) , \sqrt{\frac{5}{8} - \frac{\sqrt{5}}{8}} \} , \{ \frac{1}{4} \times ( - 1 - \sqrt{5} ) + \frac{1}{4} \times ( - 1 + \sqrt{5} ) , - \sqrt{\frac{5}{8} - \frac{\sqrt{5}}{8}} + \sqrt{\frac{5}{8} + \frac{\sqrt{5}}{8}} \} , \\ \{ \frac{1}{4} \times ( - 1 - \sqrt{5} ) + \frac{1}{2} \times ( - 1 + \sqrt{5} ) , - \sqrt{\frac{5}{8} - \frac{\sqrt{5}}{8}} + 2 \sqrt{\frac{5}{8} + \frac{\sqrt{5}}{8}} \} , \{ \frac{1}{4} \times ( - 1 - \sqrt{5} ) + \frac{3}{4} \times ( - 1 + \sqrt{5} ) , - \sqrt{\frac{5}{8} - \frac{\sqrt{5}}{8}} + 3 \sqrt{\frac{5}{8} + \frac{\sqrt{5}}{8}} \} , \\ \{ - 1 + \sqrt{5} + \frac{1}{4} \times ( - 1 - \sqrt{5} ) , - \sqrt{\frac{5}{8} - \frac{\sqrt{5}}{8}} + 4 \sqrt{\frac{5}{8} + \frac{\sqrt{5}}{8}} \} , \{ - 1 + \sqrt{5} + \frac{1}{4} \times ( - 1 - \sqrt{5} ) , + \frac{1}{4} \times ( - 1 + \sqrt{5} ) , - \sqrt{\frac{5}{8} - \frac{\sqrt{5}}{8}} + 5 \sqrt{\frac{5}{8} + \frac{\sqrt{5}}{8}} \} , \\ \{ - 1 + \sqrt{5} + \frac{1}{2} \times ( - 1 - \sqrt{5} ) , + \frac{1}{4} \times ( - 1 + \sqrt{5} ) , - 2 \sqrt{\frac{5}{8} - \frac{\sqrt{5}}{8}} + 5 \sqrt{\frac{5}{8} + \frac{\sqrt{5}}{8}} \} , , \\ \{ - 1376 + 34 \sqrt{5} + \frac{3}{4} \times ( - 1 - \sqrt{5} ) + \frac{3}{4} \times ( - 1 + \sqrt{5} ) + 84 Cos [ Null ] , - 337 \sqrt{\frac{5}{8} - \frac{\sqrt{5}}{8}} + 81 \sqrt{\frac{5}{8} + \frac{\sqrt{5}}{8}} + 84 Sin [ Null ] \} , \\ \{ - 1377 + 35 \sqrt{5} + \frac{3}{4} \times ( - 1 - \sqrt{5} ) + 84 Cos [ Null ] , - 337 \sqrt{\frac{5}{8} - \frac{\sqrt{5}}{8}} + 82 \sqrt{\frac{5}{8} + \frac{\sqrt{5}}{8}} + 84 Sin [ Null ] \} , \\ \{ - 1377 + 35 \sqrt{5} + \frac{3}{4} \times ( - 1 - \sqrt{5} ) + \frac{1}{4} \times ( - 1 + \sqrt{5} ) + 84 Cos [ Null ] , - 337 \sqrt{\frac{5}{8} - \frac{\sqrt{5}}{8}} + 83 \sqrt{\frac{5}{8} + \frac{\sqrt{5}}{8}} + 84 Sin [ Null ] \} , \\ \{ - 1377 + 35 \sqrt{5} + \frac{3}{4} \times ( - 1 - \sqrt{5} ) + \frac{1}{2} \times ( - 1 + \sqrt{5} ) + 84 Cos [ Null ] , - 337 \sqrt{\frac{5}{8} - \frac{\sqrt{5}}{8}} + 84 \sqrt{\frac{5}{8} + \frac{\sqrt{5}}{8}} + 84 Sin [ Null ] \} , \\ \{ - 1377 + 35 \sqrt{5} + \frac{3}{4} \times ( - 1 - \sqrt{5} ) + \frac{3}{4} \times ( - 1 + \sqrt{5} ) + 84 Cos [ Null ] , - 337 \sqrt{\frac{5}{8} - \frac{\sqrt{5}}{8}} + 85 \sqrt{\frac{5}{8} + \frac{\sqrt{5}}{8}} + 84 Sin [ Null ] \} , \\ \{ - 1378 + 36 \sqrt{5} + \frac{3}{4} \times ( - 1 - \sqrt{5} ) + 84 Cos [ Null ] , - 337 \sqrt{\frac{5}{8} - \frac{\sqrt{5}}{8}} + 84 \sqrt{\frac{5}{8} + \frac{\sqrt{5}}{8}} + 84 Sin [ Null ] \} , \\ \{ - 1379 + 35 \sqrt{5} + 84 Cos [ Null ] , - 336 \sqrt{\frac{5}{8} - \frac{\sqrt{5}}{8}} + 84 \sqrt{\frac{5}{8} + \frac{\sqrt{5}}{8}} + 84 Sin [ Null ] \} \}\end{matrix}$
```

```
Attachment B- MATHEMATICA CODE PRINTOUT FOR GGF ALGORITHM n=17 and
{r1,r2,r3,r4}~{8,16,24,32} INCLUDING GGF MOTIF OUTPUT SHOWN AS FIG. 7
[ANGLE DATA AND CONVERSIONS TRUNCATED DUE TO EXCESSIVE LENGTH OF
GENOME: 72 000 bp]
SetDirectory[ ]
phagegenome = Import[″C:/Users/chagan.AUMINCO/My Documents/_A_A_A_A_Drosophila/DrosdataFBGN0000179.txt″];
phageletters = Characters[phagegenome];
FormattedBases = Partition[phageletters, 14];
FourteenthBases = Drop[FormattedBases, None, 13];
FlatFourteen = Flatten[FourteenthBases];
BaseTenParts = Partition[FlatFourteen, 10];
FourteenBlocksForty = Flatten[Table[BaseTenParts, 14]];
FormattedBases = Gather[Partition[FourteenBlocksForty, 10]];
Flatten[FormattedBases];
ToExpression[%];
$Angles = ReplaceAll [ \{ a \to \frac{8 Pi}{17} , c \to \frac{16 Pi}{17} , g \to \frac{24 Pi}{17} , t \to \frac{32 Pi}{17} \} ] [ \% ]$
Map[AngleVector, Angles];
DownPhage = Accumulate[%]
(*ListPlot[DownPhage]*)
Protect[DownPhage]
ClearAll[phageletters, FormattedBases ]
phagegenome = Import[″C:/Users/chagan.AUMINCO/My Documents/_A_A_A_A_Drosophila/DrosdataFBGN0000179.txt″];
phageletters = Characters[phagegenome];
phageletters = Characters[phagegenome];
FormattedBases = Partition[phageletters, 14];
FourteenthBases = Drop[FormattedBases, None, 13];
FlatFourteen = Flatten[FourteenthBases];
BaseTenParts = Partition[FlatFourteen, 10];
FourteenBlocksForty = Flatten[Table[BaseTenParts, 14]];
FormattedBases = Gather[Partition[FourteenBlocksForty, 10]];
Flatten[FormattedBases];
ToExpression[%];
$AnglesM = ReplaceAll [ \{ t \to \frac{8 Pi}{17} , g \to \frac{16 Pi}{17} , c \to \frac{24 Pi}{17} , a \to \frac{32 Pi}{17} \} ] [ \% ]$
Map[AngleVector, AnglesM] ;
UpPhage = Accumulate [%]
ListPlot[UpPhage]
ListPlot[DownPhage]
C:\Users\chagen.AUMINCO
$\begin{matrix}\{ \frac{16 π}{17} , \frac{8 π}{17} , \frac{24 π}{17} , \frac{8 π}{17} , \frac{16 π}{17} , \frac{24 π}{17} , \frac{16 π}{17} , \frac{8 π}{17} , \frac{32 π}{17} , \frac{32 π}{17} , \frac{16 π}{17} , \frac{8 π}{17} , \frac{24 π}{17} , \frac{8 π}{17} , \frac{16 π}{17} , \frac{24 π}{17} , \frac{16 π}{17} , \frac{8 π}{17} , \frac{32 π}{17} , \frac{32 π}{17} , \frac{16 π}{17} , \frac{8 π}{17} , \frac{24 π}{17} , \frac{8 π}{17} , \\ \frac{16 π}{17} , \frac{24 π}{17} , \frac{16 π}{17} , \frac{8 π}{17} , \frac{32 π}{17} , \frac{32 π}{17} , \frac{16 π}{17} , \frac{8 π}{17} , \frac{24 π}{17} , \frac{8 π}{17} , \frac{16 π}{17} , \frac{24 π}{17} , \frac{16 π}{17} , \frac{8 π}{17} , \frac{32 π}{17} , \frac{32 π}{17} , \frac{16 π}{17} , \frac{8 π}{17} , \frac{24 π}{17} , \frac{8 π}{17} , \frac{16 π}{17} , \frac{24 π}{17} , \frac{16 π}{17} , \frac{8 π}{17} , \\ \frac{32 π}{17} , \frac{32 π}{17} , \frac{16 π}{17} , \frac{8 π}{17} , \frac{24 π}{17} , \frac{8 π}{17} , \frac{16 π}{17} , \frac{24 π}{17} , \frac{16 π}{17} , \frac{4 π}{17} , \frac{32 π}{17} , \frac{32 π}{17} , , \frac{8 π}{17} , \frac{8 π}{17} , \frac{16 π}{17} , \frac{8 π}{17} , \frac{32 π}{17} , \frac{24 π}{17} , \frac{8 π}{17} , \frac{32 π}{17} , \frac{8 π}{17} , \frac{8 π}{17} , \frac{8 π}{17} , \\ \frac{8 π}{17} , \frac{16 π}{17} , \frac{8 π}{17} , \frac{32 π}{17} , \frac{24 π}{17} , \frac{8 π}{17} , \frac{32 π}{17} , \frac{8 π}{17} , \frac{8 π}{17} , \frac{8 π}{17} , \frac{8 π}{17} , \frac{16 π}{17} , \frac{8 π}{17} , \frac{32 π}{17} , \frac{24 π}{17} , \frac{8 π}{17} , \frac{32 π}{17} , \frac{8 π}{17} , \frac{8 π}{17} , \frac{8 π}{17} , \frac{8 π}{17} , \frac{16 π}{17} , \frac{8 π}{17} , \frac{32 π}{17} , \frac{24 π}{17} , \\ \frac{8 π}{17} , \frac{32 π}{17} , \frac{8 π}{17} , \frac{8 π}{17} , \frac{8 π}{17} , \frac{8 π}{17} , \frac{16 π}{17} , \frac{8 π}{17} , \frac{32 π}{17} , \frac{24 π}{17} , \frac{8 π}{17} , \frac{32 π}{17} , \frac{8 π}{17} , \frac{8 π}{17} , \frac{8 π}{17} , \frac{8 π}{17} , \frac{16 π}{17} , \frac{8 π}{17} , \frac{32 π}{17} , \frac{24 π}{17} , \frac{8 π}{17} , \frac{32 π}{17} , \frac{8 π}{17} \}\end{matrix}$
    large output  show less  show more  show all  set size limit . . .
$\begin{matrix}\{ \{ - Cos [ \frac{π}{17} ] , Sin [ \frac{π}{17} ] \} , \{ - Cos [ \frac{π}{17} ] + Sin [ \frac{π}{34} ] , Cos [ \frac{π}{34} ] + Sin [ \frac{π}{17} ] \} , \{ - Cos [ \frac{π}{17} ] + Sin [ \frac{π}{34} ] - Sin [ \frac{3 π}{34} ] , Cos [ \frac{π}{34} ] - Cos [ \frac{3 π}{34} ] + Sin [ \frac{π}{17} ] \} , \\ \{ - Cos [ \frac{π}{17} ] + 2 Sin [ \frac{π}{34} ] - Sin [ \frac{3 π}{34} ] , 2 Cos [ \frac{π}{34} ] - Cos [ \frac{3 π}{34} ] + Sin [ \frac{π}{17} ] \} , , \\ \{ 1204 Cos [ Null ] - 15 \; 148 Cos [ \frac{π}{17} ] + 21 \; 909 Cos [ \frac{2 π}{17} ] + 20 \; 355 Sin [ \frac{π}{34} ] - 15 \; 302 Sin [ \frac{3 π}{34} ] , 20 \; 355 Cos [ \frac{π}{34} ] - 15 \; 302 Cos [ \frac{3 π}{34} ] + \\ 1204 Sin [ Null ] + 15 \; 148 Sin [ \frac{π}{17} ] - 21 \; 909 Sin [ \frac{2 π}{17} ] \} , \\ \{ 1204 Cos [ Null ] - 15 \; 148 Cos [ \frac{π}{17} ] + 21 \; 910 Cos [ \frac{2 π}{17} ] + 20 \; 355 Sin [ \frac{π}{34} ] - 15 \; 302 Sin [ \frac{3 π}{34} ] , 20 \; 355 Cos [ \frac{π}{34} ] - 15 \; 302 Cos [ \frac{3 π}{34} ] + \\ 1204 Sin [ Null ] + 15 \; 148 Sin [ \frac{π}{17} ] - 21 \; 910 Sin [ \frac{2 π}{17} ] \} , \\ \{ 1204 Cos [ Null ] - 15 \; 148 Cos [ \frac{π}{17} ] + 21 \; 910 Cos [ \frac{2 π}{17} ] + 20 \; 356 Sin [ \frac{π}{34} ] - 15 \; 302 Sin [ \frac{3 π}{34} ] , 20 \; 356 Cos [ \frac{π}{34} ] - 15 \; 302 Cos [ \frac{3 π}{34} ] + \\ 1204 Sin [ Null ] + 15 \; 148 Sin [ \frac{π}{17} ] - 21 \; 910 Sin [ \frac{2 π}{17} ] \} \}\end{matrix}$
    large output  show less  show more  show all  set size limit . . .
$\begin{matrix}\{ \frac{24 π}{17} , \frac{32 π}{17} , \frac{14 π}{17} , \frac{24 π}{17} , \frac{24 π}{17} , \frac{24 π}{17} , \frac{24 π}{17} , \frac{32 π}{17} , \frac{3 π}{17} , \frac{9 π}{17} , \frac{24 π}{17} , \frac{32 π}{17} , \frac{16 π}{17} , \frac{24 π}{17} , \frac{24 π}{17} , \frac{16 π}{17} , \frac{24 π}{17} , \frac{24 π}{17} , \frac{8 π}{17} , \frac{9 π}{17} , \frac{24 π}{17} , \frac{32 π}{17} , \frac{24 π}{17} , \\ \frac{32 π}{17} , \frac{24 π}{17} , \frac{16 π}{17} , \frac{24 π}{17} , \frac{28 π}{17} , \frac{8 π}{17} , \frac{9 π}{17} , \frac{24 π}{17} , \frac{32 π}{17} , \frac{16 π}{17} , \frac{32 π}{17} , \frac{24 π}{17} , \frac{16 π}{17} , \frac{24 π}{17} , \frac{32 π}{17} , \frac{6 π}{17} , \frac{8 π}{17} , \frac{24 π}{17} , \frac{32 π}{17} , \frac{16 π}{17} , \frac{32 π}{17} , \frac{24 π}{17} , \frac{16 π}{17} , \\ \frac{24 π}{17} , \frac{32 π}{17} , \frac{6 π}{17} , \frac{8 π}{17} , \frac{24 π}{17} , \frac{32 π}{17} , \frac{16 π}{17} , \frac{32 π}{17} , \frac{24 π}{17} , \frac{16 π}{17} , \frac{24 π}{17} , , \frac{24 π}{17} , \frac{32 π}{17} , \frac{8 π}{17} , \frac{16 π}{17} , \frac{32 π}{17} , \frac{8 π}{17} , \frac{32 π}{17} , \frac{32 π}{17} , \frac{32}{17} , \frac{32 π}{17} , \frac{24 π}{17} , \\ \frac{32 π}{17} , \frac{8 π}{17} , \frac{16 π}{17} , \frac{32 π}{17} , \frac{8 π}{17} , \frac{32 π}{17} , \frac{32 π}{17} , \frac{32 π}{17} , \frac{32 π}{17} , \frac{24 π}{17} , \frac{32 π}{17} , \frac{8 π}{17} , \frac{16 π}{17} , \frac{32 π}{17} , \frac{8 π}{17} , \frac{32 π}{17} , \frac{32 π}{17} , \frac{32 π}{17} , \frac{32 π}{17} , \frac{24 π}{17} , \frac{32 π}{17} , \frac{8 π}{17} , \frac{16 π}{17} , \\ \frac{32 π}{17} , \frac{8 π}{17} , \frac{32 π}{17} , \frac{32 π}{17} , \frac{32 π}{17} , \frac{32 π}{17} , \frac{24 π}{17} , \frac{32 π}{17} , \frac{8 π}{17} , \frac{16 π}{17} , \frac{32 π}{17} , \frac{8 π}{17} , \frac{32 π}{17} , \frac{32 π}{17} , \frac{32 π}{17} , \frac{32 π}{17} , \frac{24 π}{17} , \frac{32 π}{17} , \frac{8 π}{17} , \frac{16 π}{17} , \frac{32 π}{17} , \frac{8 π}{17} , \frac{32 π}{17} \}\end{matrix}$
    large output  show less  show more  show all  set size limit . . .
$\begin{matrix}\{ \{ - Sin [ \frac{3 π}{34} ] , - Cos [ \frac{3 π}{34} ] \} , \{ Cos [ \frac{2 π}{17} ] - Sin [ \frac{3 π}{34} ] , - Cos [ \frac{3 π}{34} ] - Sin [ \frac{2 π}{17} ] \} , \{ - Cos [ \frac{π}{17} ] + Cos [ \frac{2 π}{17} ] - Sin [ \frac{3 π}{34} ] , - Cos [ \frac{3 π}{34} ] , + \\ Sin [ \frac{π}{17} ] + Sin [ \frac{2 π}{17} ] \} , \{ - Cos [ \frac{π}{17} ] + 2 Cos [ \frac{2 π}{17} ] - Sin [ \frac{3 π}{34} ] , - Cos [ \frac{3 π}{34} ] + Sin [ \frac{π}{17} ] - 2 Sin [ \frac{3 π}{17} ] \} , , \\ \{ 1204 Cos [ Null ] - 15 \; 302 Cos [ \frac{π}{17} ] + 20 \; 355 Cos [ \frac{2 π}{17} ] + 21 \; 909 Sin [ \frac{π}{34} ] - 15 \; 148 Sin [ \frac{3 π}{34} ] , 21 \; 909 Cos [ \frac{π}{34} ] - 15 \; 148 Cos [ \frac{3 π}{34} ] + \\ 1204 Sin [ Null ] + 15 \; 302 Sin [ \frac{π}{17} ] - 20 \; 355 Sin [ \frac{2 π}{17} ] \} , \\ \{ 1204 Cos [ Null ] - 15 \; 302 Cos [ \frac{π}{17} ] + 20 \; 355 Cos [ \frac{2 π}{17} ] + 21 \; 910 Sin [ \frac{π}{34} ] - 15 \; 148 Sin [ \frac{3 π}{34} ] , 21 \; 910 Cos [ \frac{π}{34} ] - 15 \; 148 Cos [ \frac{3 π}{34} ] + \\ 1204 Sin [ Null ] + 15 \; 302 Sin [ \frac{π}{17} ] - 20 \; 355 Sin [ \frac{2 π}{17} ] \} , \\ \{ 1204 Cos [ Null ] - 15 \; 302 Cos [ \frac{π}{17} ] + 20 \; 356 Cos [ \frac{2 π}{17} ] + 21 \; 910 Sin [ \frac{π}{34} ] - 15 \; 148 Sin [ \frac{3 π}{34} ] , 21 \; 910 Cos [ \frac{π}{34} ] - 15 \; 148 Cos [ \frac{3 π}{34} ] + \\ 1204 Sin [ Null ] + 15 \; 302 Sin [ \frac{π}{17} ] - 20 \; 356 Sin [ \frac{2 π}{17} ] \} \}\end{matrix}$
```

What is claimed is:

1. A sequence analysis method for analysing sequences of encoding elements of a particular DNA, RNA, or macromolecule sequence, comprising the steps of:
providing a sequence data file defining an ordered collection of encoding elements, each being one of a plurality of encoding element types;
formatting the sequence data file to generate a formatted data file, wherein the formatted data file corresponds to a representation of the sequence data file according to one or more user-defined and/or pre-defined formatting parameters, the formatted data file defining an ordered set of encoding elements;
determining an angle set defining, for each encoding element type, a corresponding angle in an n-dimensional space (n>1), wherein each angle may be defined in polar co-ordinates, the determination based on one or more user-defined and/or pre-defined angle generation parameters;
recursively and in order, applying the angle set to the formatted data file, thereby generating a mapped data file, said mapped data file defining a set of points in the n-dimensional space and linkages between adjacent pairs of points;
displaying and/or storing the mapped data file, wherein the mapped data file is configured to enable generation for display of a visual representation of the relative locations of the points in the n-dimensional space and the associated linkages.

2. A sequence generation method for generating sequences of DNA, RNA, or macromolecule encoding elements, each being one of a plurality of encoding element types, comprising the steps of:
providing a spatial data file defining a measured or desired spatial representation of a biological sample;
determining a profile of the spatial representation in an n-dimensional space (n>1) according to one or more user-defined and/or pre-defined profile parameters;
determining an angle set defining, for each encoding element type, a corresponding angle in an n-dimensional space (n>1), wherein each angle may be defined in polar co-ordinates, the determination based on one or more user-defined and/or pre-defined angle generation parameters;
utilising the angle set to identify a predictive data file defining sequence of encoding elements, wherein an initial position in the profile is selected and an outline of said profile is generated by recursively identifying particular encoding elements based on a best-fit identification of a next angle selected from the angle set such as to optimise a similarity between the profile and the outline; and
storing the predictive data file.

3. A method of analysing a generated sequence, comprising the steps of:
providing spatial data file defining a measured or desired spatial representation of a biological sample
performing the method of claim 2 to the spatial data file to generate a predictive data file; and
performing the method of claim 1, wherein the provided sequence data file is the predictive data file.


SEQUENCE LISTING (15 sequences)

SEQ ID NO: 1 - DNA, 77 bp, synthetic construct
  source 1..77: mol_type = other DNA
gatcgactagtcatctaatccgtgcaaagtgttcccaattgggcgtgcatgctgttcccatacgtttggattaagtg

SEQ ID NO: 2 - DNA, 24 bp, synthetic construct
  source 1..24: mol_type = other DNA
tagtagcttatgctgaacgtgtgc

SEQ ID NO: 3 - DNA, 10 bp, synthetic construct
  source 1..10: mol_type = other DNA
tagtagctta

SEQ ID NO: 4 - DNA, 10 bp, synthetic construct
  source 1..10: mol_type = other DNA
tgctgaacgt

SEQ ID NO: 5 - DNA, 60 bp, synthetic construct
  source 1..60: mol_type = other DNA
tagtagcttatagtagcttatagtagcttatagtagcttatagtagcttatagtagctta

SEQ ID NO: 6 - DNA, 50 bp, synthetic construct
  source 1..50: mol_type = other DNA
tgctgaacgttgctgaacgttgctgaacgttgctgaacgttgctgaacgt

SEQ ID NO: 7 - DNA, 10 bp, synthetic construct
  source 1..10: mol_type = other DNA
tagtacgtta

SEQ ID NO: 8 - DNA, 10 bp, synthetic construct
  source 1..10: mol_type = other DNA
gaaaaaggga

SEQ ID NO: 9 - DNA, 15 bp, synthetic construct
  source 1..15: mol_type = other DNA
aaagaaaaagggaaa

SEQ ID NO: 10 - protein, 9 aa, synthetic construct
  SITE 1: note = Pyroglutamic acid
  SITE 6: note = D-Amino acid
  SITE 9: note = Ethylamide acetic acid modified residue
XHWSYLLRP

SEQ ID NO: 11 - protein, 10 aa, synthetic construct
  SITE 1: note = Phosphorylated residue
  SITE 2: note = Phosphorylated residue
  SITE 3: note = Phosphorylated residue
  SITE 4: note = Phosphorylated residue
  SITE 5: note = Phosphorylated residue
  SITE 6: note = Tert-butyl D-serine
  SITE 10: note = Azaglycine amide
EHWSYSLRPX

SEQ ID NO: 12 - protein, 8 aa, synthetic construct
  SITE 2: note = D-Amino acid
  SITE 5: note = D-Amino acid
  SITE 8: note = Cysteine thiolacetate
  DISULFID 3..8: note = Intrachain disulfide bond
HFCFWKTC

SEQ ID NO: 13 - protein, 39 aa, synthetic construct
HGEGTFTSDLSKQMEEEAVRLFIEWLKNGGPSSGAPPPS

SEQ ID NO: 14 - protein, 34 aa, synthetic construct
SVSEIQLMHNLGKHLNSMERVEWLRKKLQDVHNF

SEQ ID NO: 15 - protein, 35 aa, synthetic construct
  SITE 1: note = Acetylated residue
  SITE 35: note = Amidated residue
YTSLIHSLIEESQQQELNEQELLELDKWASLWNWF